(19) United States
(12) Patent Application Publication
(10) Pub. No.: US 2007/0219557 A1
(43) Pub. Date: Sep. 20, 2007

(54) Soft tissue fixation

(76) Inventors: Bernard J. Bourque, Taunton, MA (US); Michael C. Ferragamo, Foster, RI (US); Sally L. Carter, N. Attleboro, MA (US); George Sikora, Bridgewater, MA (US)

Correspondence Address: NORMAN F. HAINER, JR.;SMITH & NEPHEW, INC., 150 MINUTEMAN ROAD, ANDOVER, MA, 01801, US

(21) Appl. No.: 11/377,666

(22) Filed: Mar. 17, 2006

(51) Int. Cl. A61B 17/58

(52) U.S. Cl. 606/72

(57) ABSTRACT

A surgical device includes an anchor body defining a cavity and an opening to the cavity, and a flexible member extending through the opening and having a sliding knot disposed within the cavity. The flexible member is movable through the anchor body in a first direction while movement of the flexible member in a second, opposite direction is restricted. The flexible member forms a loop portion, a tension portion, and the sliding knot. The surgical device includes an outer shaft defining a longitudinally slot, and an inner shaft defining a recess. The flexible member passes through the recess and the slot. A method includes placing an anchor having a flexible member coupled thereto in the form of a loop in bone, passing the loop through an opening in tissue, positioning the loop around the anchor, and tensioning the flexible member to decrease the loop size.

### BACKGROUND

This description relates to soft tissue fixation.

Soft tissues, such as ligaments and tendons, can become torn or detached from bone. The tear or detachment can be repaired using sutures. It is known to repair soft tissue by inserting an anchor with attached suture into bone, and knotting the suture to secure the soft tissue to the bone.

### SUMMARY

In one general aspect, a surgical device includes an anchor body defining a cavity and an opening to the cavity, and a flexible member, for example, a suture, extending through the opening and having a sliding knot disposed within the cavity.

Embodiments of this aspect may include one or more of the following features.

The anchor body includes a portion defining the opening and the cavity, and a plug received by the portion. The sliding knot is, for example, a slip knot or other knot such that the flexible member is movable through the anchor body in a first direction while movement of the flexible member in a second, opposite direction is restricted. The opening is a through hole, and the anchor body defines at least two or three through holes. The anchor body defines a groove in an exterior surface of the anchor body contiguous with the opening. The anchor body includes at least one thread extending from the exterior surface for rotary advancement (e.g., by screwing) into a target tissue. Alternatively, the anchor body includes at least one circumferential rib for axially-oriented advancement (e.g., by tapping) into a target tissue.

The flexible member forms a loop portion, a tension portion, and the sliding knot.

In an illustrated embodiment, the surgical device includes an outer shaft in which the anchor body is received, and an inner shaft received within the outer shaft. The surgical device includes a handle, and the outer shaft is attached to or movable with respect to the handle. The inner shaft is arranged for engagement with a proximal portion of the anchor.

In another general aspect, a surgical device includes an outer shaft defining a longitudinally extending slot, and an inner shaft received within the outer shaft. The inner shaft defines a recess associated with the slot of the outer shaft such that when an anchor with a flexible member coupled thereto is received within the outer shaft, the flexible member passes through the recess and the slot.

Embodiments of this aspect may include the anchor.

In another general aspect, repairing soft tissue includes placing an anchor having a flexible member coupled thereto in the form of a loop in bone, passing the loop through an opening in the tissue, positioning the loop around the anchor, and tensioning the flexible member to decrease a size of the loop to secure the tissue to the bone.

Embodiments of this aspect may include one or more of the following features.

Positioning the loop includes passing the anchor through the loop and into the bone. Alternatively, positioning the loop occurs after placing the anchor.

In another general aspect, a tissue fixation member for use in a surgical procedure includes a body, a flexible member, and means for coupling the flexible member to the anchor. The tissue fixation member is configured such that without having to tie a knot in the flexible member during the surgical procedure, the flexible member is movable through the body in a first direction and movement of the flexible member in a second, opposite direction is restricted.

In another general aspect, a surgical device includes an outer shaft defining a slot, an inner shaft positioned within the outer shaft, and an anchor received within the outer shaft. The anchor includes having a portion forming a loop extending through the slot. The inner shaft is arranged to impart an advancement force to the anchor.

One or more of the foregoing embodiments may include one or more of the following advantages. Repair of torn or damaged tissue can be efficiently performed by a surgeon without the need for the surgeon to tie knots. For example, torn or damaged tissue in the vicinity of a joint, such as a ball and socket joint in a glenoid-humeral or hip joint, can be repaired without the surgeon having to tie knots. The knot can be protected from any debris or damage during insertion of an anchor and/or after the anchor is inserted in tissue. An anchor is less likely to be dislodged from a repair site if the knot is contained within or protected by the anchor body. An anchor can be provided with a knot that has been tied in advance, e.g., the surgeon can insert the anchor and secure the anchor in place without having to tie any knots within the vicinity or confines of a tissue repair site.

The details of one or more embodiments are set forth in the accompanying drawings and the description below. Other features, objects, and advantages will be apparent from the description and drawings, and from the claims.

### DESCRIPTION OF DRAWINGS

FIG. 1 is a partial sectional view of an anchor securing a torn labrum to a glenoid of a shoulder joint.

FIG. 2 is a perspective view of an anchor delivery device and anchor according to a first embodiment.

FIG. 3 is a perspective view of a an anchor according to the first embodiment.

FIG. 4 is an exploded, perspective view of the anchor of FIG. 3.

FIG. 5 is a sectional view of the anchor taken along line 5-5 in FIG. 4.

FIG. 6A is a sectional view of the anchor of FIG. 3 coupled with a suture.

FIG. 6B shows the formation of a sliding knot within a cavity of the anchor of FIG. 3.

FIG. 7 is an exploded, perspective view of the anchor delivery device of FIG. 2.

FIG. 8 is a view of a proximal portion of the anchor delivery device of FIG. 2.

FIG. 9 is a sectional view of the proximal portion of the anchor delivery device of FIG. 8.

FIGS. 10-21 show the anchor delivery device and anchor of FIG. 2 in use.

FIGS. 22-26 show an alternative use of the anchor delivery device and anchor of FIG. 2.

FIG. 27A is a perspective view of an anchor delivery device and anchor according to another embodiment.

FIG. 27B is a sectional view of the anchor delivery device and anchor of FIG. 27A.

FIG. 28A is a perspective view of a distal portion of the anchor delivery device and the anchor of FIG. 27A.

FIG. 28B is a sectional view of the distal portion of the anchor delivery device and the anchor of FIG. 28A.

FIG. 28C-28D are sectional views of the proximal portion of anchor bodies for use with the anchor delivery device of FIG. 27A.

FIG. 29 is a perspective view of the anchor of FIG. 28.

FIG. 30 is a perspective view of an anchor delivery device and anchor according to another embodiment.

FIG. 31 is a sectional view of the anchor delivery device and anchor of FIG. 30.

FIG. 32 is a sectional view of the anchor delivery device of FIG. 30.

FIG. 33A is a partial sectional view of the anchor delivery device and anchor of FIG. 30 in a first extended position.

FIG. 33B is a partial side view of a handle and hub portion of the anchor delivery device of FIG. 33A.

FIG. 34A is a partial sectional view of the anchor delivery device and anchor of FIG. 30 in a second, retracted position.

FIG. 34B is a partial side view of a handle and hub portion of the anchor delivery device of FIG. 34A.

FIG. 35 is a partial sectional view of the handle and hub portion of the anchor delivery device of FIG. 33B.

FIG. 36 is a perspective view of a distal portion of the anchor delivery device of FIG. 31 shown in the second, retracted position.

FIG. 37A is a perspective view of an anchor and anchor delivery device according to another embodiment.

FIG. 37B is an exploded perspective view of the anchor and anchor delivery device of FIG. 36.

FIG. 37C is an enlarged view of a distal portion of the anchor delivery device of FIG. 37B.

FIG. 38A is perspective view of the anchor of FIG. 36.

FIGS. 38B-C are partial sectional views of the distal portion of the anchor and anchor delivery device of FIG. 36.

FIG. 39 is a sectional view of the anchor of FIG. 38.

FIG. 40 is a sectional view of an anchor delivery device.

FIG. 41 are enlarged plan views of the suture puller device of FIG. 7.

FIG. 42A is a perspective view of an anchor according to another embodiment.

FIG. 42B is a perspective view of the anchor of FIG. 42B and an anchor delivery device.

### DETAILED DESCRIPTION

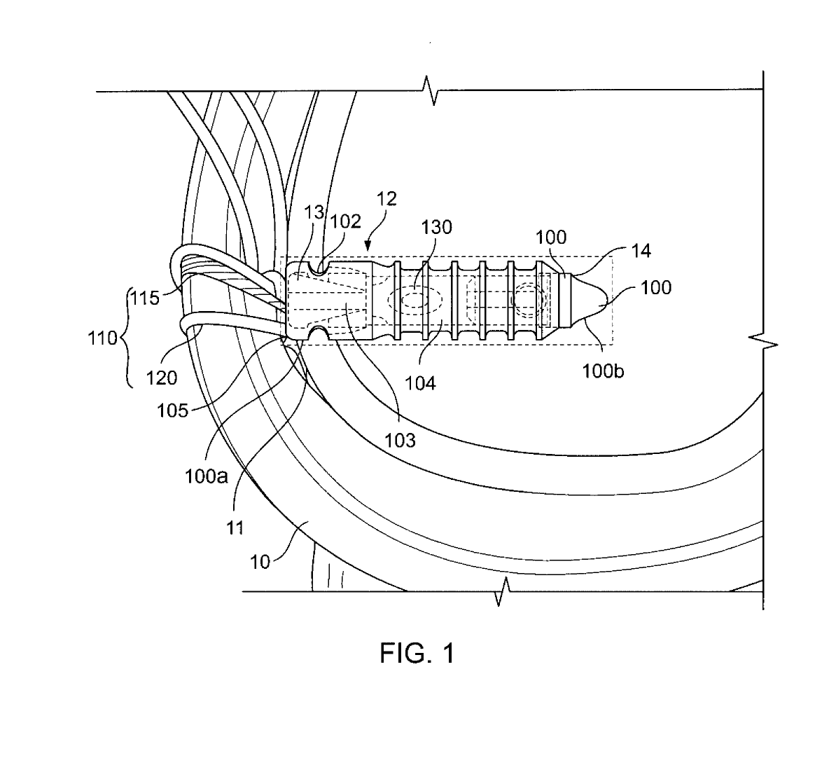

Referring to FIG. 1, a glenoid labrum 10 having a tear 11 is secured with an anchor 100 to a bony surface 13 of a glenoid 12 of a patient's shoulder. The anchor 100 is positioned within a hole 14 drilled within the glenoid 12, and the tear 11 is secured to the glenoid with a suture 110 of the anchor. The anchor 100 has an elongated body 107 defining a hollow cavity 104 and three suture holes 101, 102, 103 extending from the hollow cavity 104 to an exterior surface 105 of a proximal portion 100a of the anchor 100. Suture 110 has a tensioning portion 115 extending through centrally located hole 103, and a loop portion 120 extending through angled side holes 101, 102. As described below, the suture is threaded through holes 101, 102 and 103 such that pulling on tensioning portion 115 shortens the length of loop portion 120 to secure the soft tissue to the bone. The suture 110 has a sliding knot 130 positioned within the anchor cavity 104 that allows the length of loop portion 120 to be shortened, but limits lengthening of loop portion 120.

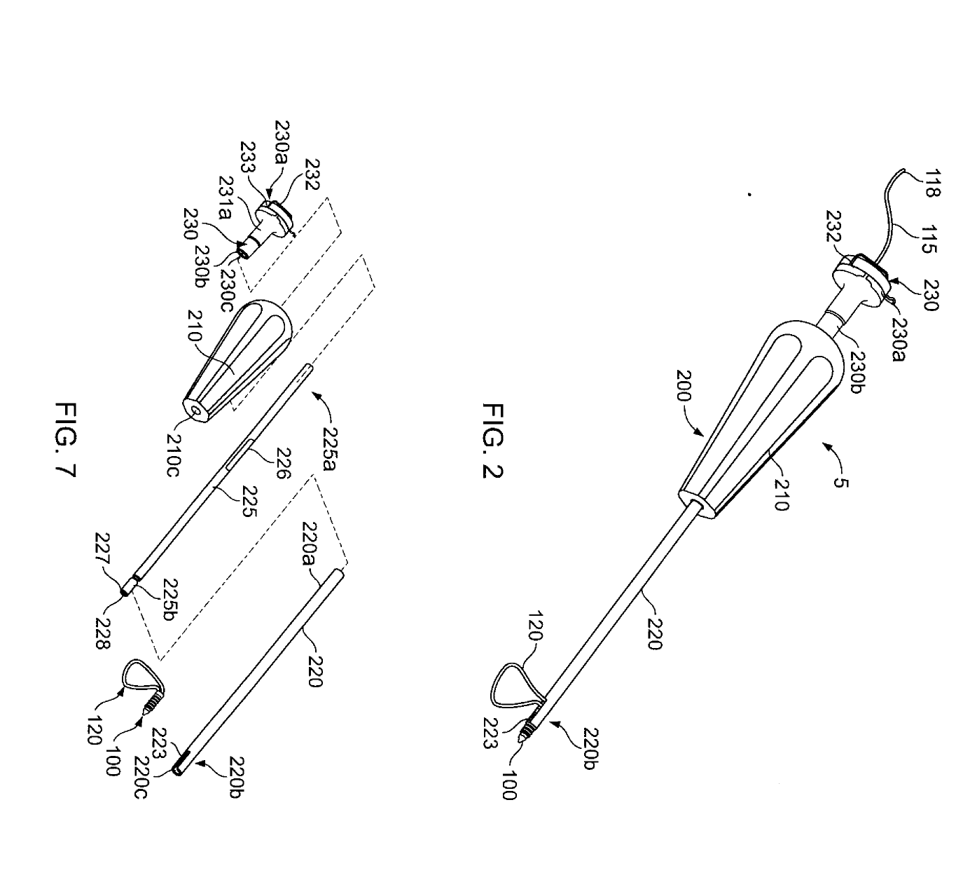

Referring to FIG. 2, a surgical device 5 for securing soft tissue to bone includes the anchor 100 and an anchor delivery device 200. The anchor delivery device 200 includes a handle 210, an outer shaft 220 extending distally from the handle 210, and a suture puller 230 extending proximally from the handle 210. The anchor 100 is loaded within a distal portion 220b of the outer shaft 220.

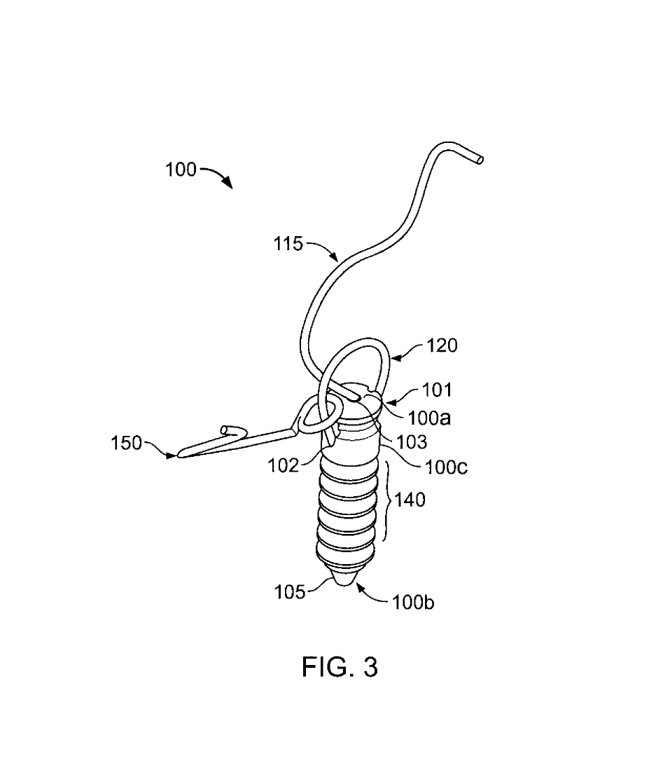

Referring to FIG. 3, the anchor 100, such as a bone anchor for axially-oriented advancement into tissue, e.g., by tapping, includes non-helical, circumferential extending ribs 140 extending outwardly from the exterior surface 100c of the anchor 100 between the proximal portion 100a and a distal portion 100b of the anchor. The ribs 140 secure the anchor 100 to the interior walls of a pre-drilled hole within a bony surface, such as in the glenoid 12. The distal portion 100b of the anchor 100 includes a tapered tip 105 for ease of delivery into the pre-drilled hole. Coupled to the loop portion 120 of the suture 110 is a leader or passing suture 150 for purposes described below.

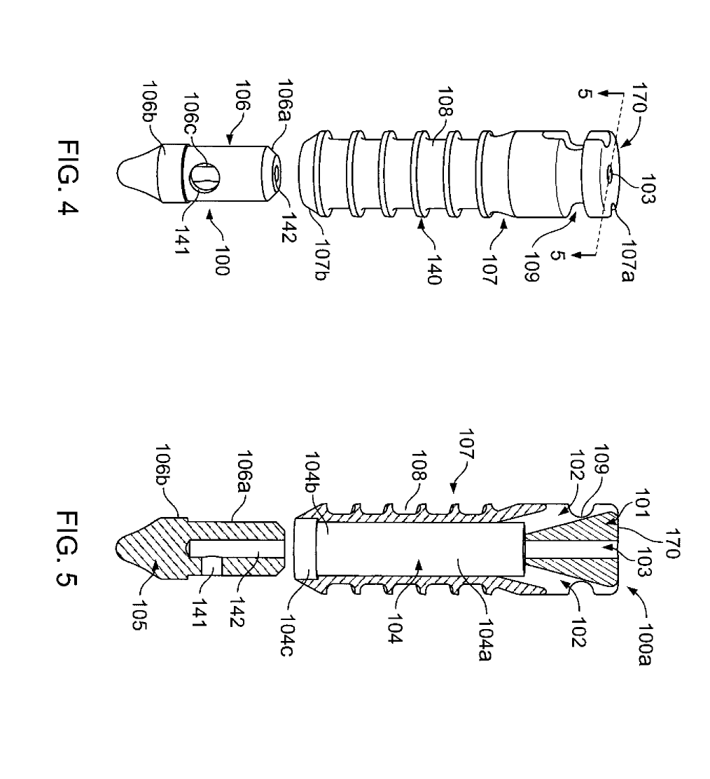

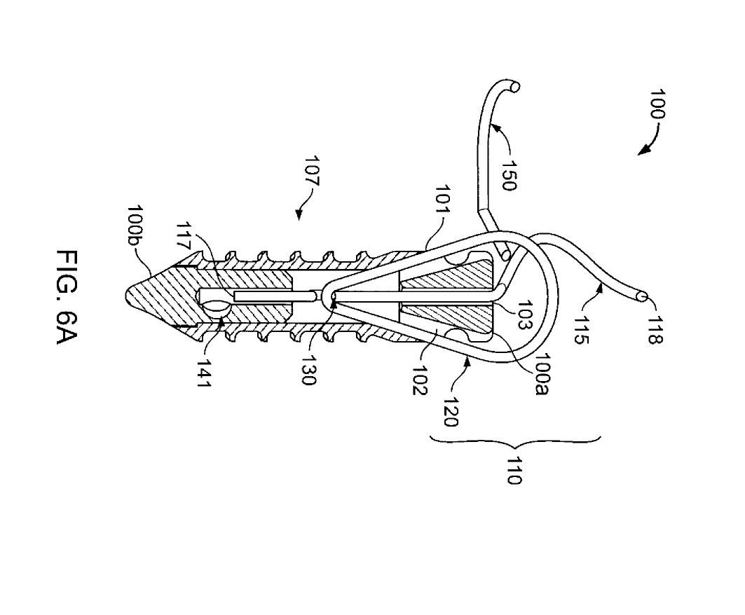

Referring to FIGS. 4 and 5, to facilitate threading and knotting of the suture 110, the anchor body 107 is formed from two pieces, a plug 106 and an anchoring portion 108. The anchoring portion 108 includes a circumferential groove 109 at the proximal portion 100a in which the suture 110 extending from hole 103 can be positioned for tensioning the suture 110 during a repair procedure. The anchoring portion 108 defines the centrally located hole 103 extending from the cavity 104 to the proximal face 170 of the anchoring portion 108, and the suture loop holes 101, 102, one on either side of hole 103 extending angularly outward from the cavity 104 and terminating at the groove 109. The cavity 104 includes a central lumen 104a substantially cylindrical in shape and extending to a distal opening 104b to receive the plug 106. The cavity 104 includes a circumferentially extending stepped portion 104c having a diameter larger than the remainder of the central lumen 104a of the hollow cavity 104. The plug 106 includes a closed-end suture channel 142 extending distally from a proximal end 106a of the plug, and a suture hole 141 formed through a wall of the plug 160 from an exterior surface 106c of the plug to the suture channel 142. The plug 106 also includes a raised edge 106b having a diameter slightly less than the stepped portion 104c of the hollow cavity 104. The anchoring portion 108 and plug 106 are press-fit together by inserting the proximal end 106a of the plug 106 into the anchor cavity 104 after the suture 110 (not shown) is secured to the plug 106 at the suture hole 141, as described below. The raised edge 106b of the plug 106 abuts against the stepped portion 104c when the plug 106 is inserted into the hollow cavity 104.

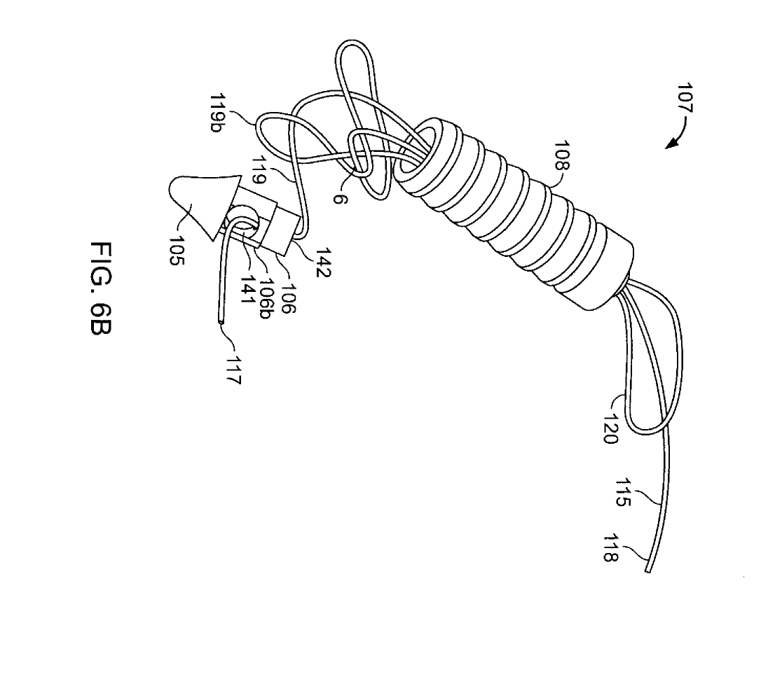

Referring to FIGS. 6A and 6B, to form the tensioning portion 115, the loop portion 120 and the sliding knot 130, a single length of suture 110 is threaded through the holes 101, 102, and 103 before positioning the plug 106 in the anchoring portion 108. During assembly of the anchor 100, the assembler feeds two end portions 117, 118 of the suture 110 through the holes 101, 102, one end through each hole to form the loop portion 120, through the cavity 104 and out the distal opening 104b. The assembler then loops one end portion 118 of the suture 110 under and then over the other end portion 117, under and then over itself to form a loop 119b, then over and under the other end portion, under itself, and then through the loop 119b to form the sliding knot 130, for example, a slip knot. The assembler then feeds the end portion 118 back through the cavity 140 and out hole 103 to form tensioning portion 115. The other end portion 117 is secured to the plug 106, for example, by passing the other end portion 117 through the channel 142 and the hole 141 and tying a knot in the end portion larger than the hole or by capturing end portion 117 between the plug 106 and the anchoring portion 108 when the plug is pres-fit into the anchoring portion, e.g., between the stepped portion 104c and the raised edge 106b.

Referring to FIGS. 2 and 7, the handle 210 defines a lumen 210c for receiving a proximal portion 220a of the outer shaft 220 by, for example, a press fit, and for receiving a distal end 230b of the suture puller 230. Outer shaft 220 defines a lumen 220c for receiving a hollow inner shaft 225. Suture puller 230 defines a lumen 230c for receiving the proximal portion of the inner shaft 225a. Suture tensioning portion 115 extends from anchor 100 through the inner shaft 225, through the lumen 230c in the suture puller 230, and out the proximal end 230a of the suture puller. The distal end 230b suture puller 230 receives the proximal end 225a of the inner shaft 225 with an interference fit and is coupled thereto for imparting an advancement force, such as an axially-oriented tapping advancement force, to the inner shaft 225 which is transferred to the anchor 100. The inner shaft 225 includes a longitudinally flat portion 226 forming a keyway for engaging a protrusion formed within the lumen 220c of the outer shaft 220. The keyway permits the inner shaft 225 and the outer shaft 220 to be coupled together in a manner that permits axial movement of the inner shaft 225, but restricts rotary movement of the inner shaft 225 relative to the outer shaft 220.

The distal portion 220b of the outer shaft 220 defines a longitudinally extending slot 223 sized and shaped for receiving the suture 110, e.g., the loop portion 220, of the anchor 100. Referring to FIG. 2, the loop portion 120 protruding from the proximal portion 100a of the anchor 100 extends through the slot 223 to maintain the loop portion 120 in a position accessible to the surgeon when the inner shaft 225 is moved axially relative to the fixed outer shaft 220. A length of the slot 223 in the axial direction is sized to permit the loop portion 120 to move freely throughout the range of movement of the inner shaft 225, e.g., to limit the loop portion 120 from becoming pinched between the inner shaft 225 and stationary outer shaft 220. The inner shaft 225 includes a distal portion 225b having a driving end 228. The driving end 228 of the inner shaft 225 engages the proximal portion 100a of the anchor 100 to impart the advancement force thereto. The distal portion 225b of the inner shaft 225 also includes a recess 227 sized for receiving the suture 110 extending from the proximal portion 100a of the anchor 110 and for limiting the suture 110 from becoming pinched between the end 228 of the movable inner shaft 225 and the proximal portion 100a of the anchor 100.

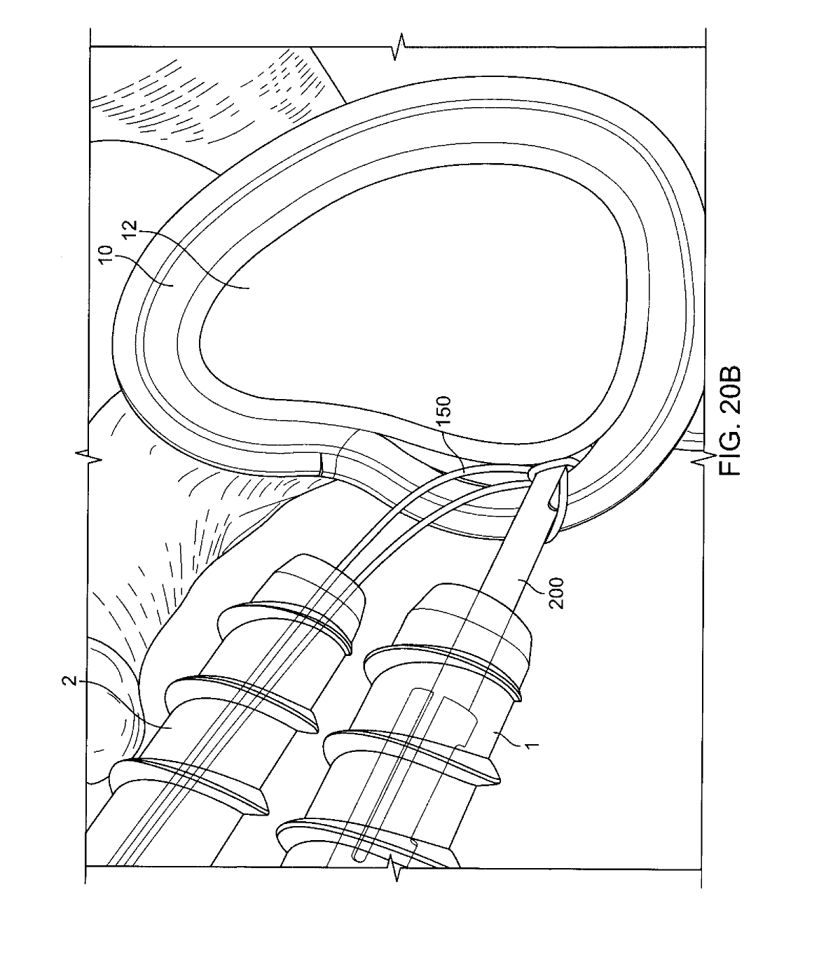

The suture puller 230 includes a proximal portion 230a having a head 233 and a distal portion 230b. The suture puller 230 includes a shaft 231 extending distally from the head 233. The shaft 231 includes a depth stop 231a having a diameter greater than a distal portion 231b of the shaft 231, e.g., the depth stop 231a is a raised or stepped edge extending circumferentially around the shaft 231 for limiting axial movement of the suture puller 230 (and inner shaft 225) to a predetermined distance, e.g., to a desired anchor depth within a pre-drilled hole. The head 233 of the suture puller 230 includes a groove 232 for securing the leading end 118 of the tensioning portion 115 of the suture 110 in an accessible position during delivery of the anchor 100. The groove 232 is sized and shaped for providing an interference fit with the tensioning portion 115 of the suture (as described below with respect to FIG. 20a) which is passed through the inner shaft 225, handle 210 and suture puller 230, and secured within the groove 232. The head 233 is also provided with a hard, flat surface 233a which can be struck with a surgical mallet to impart the tapping advancement force.

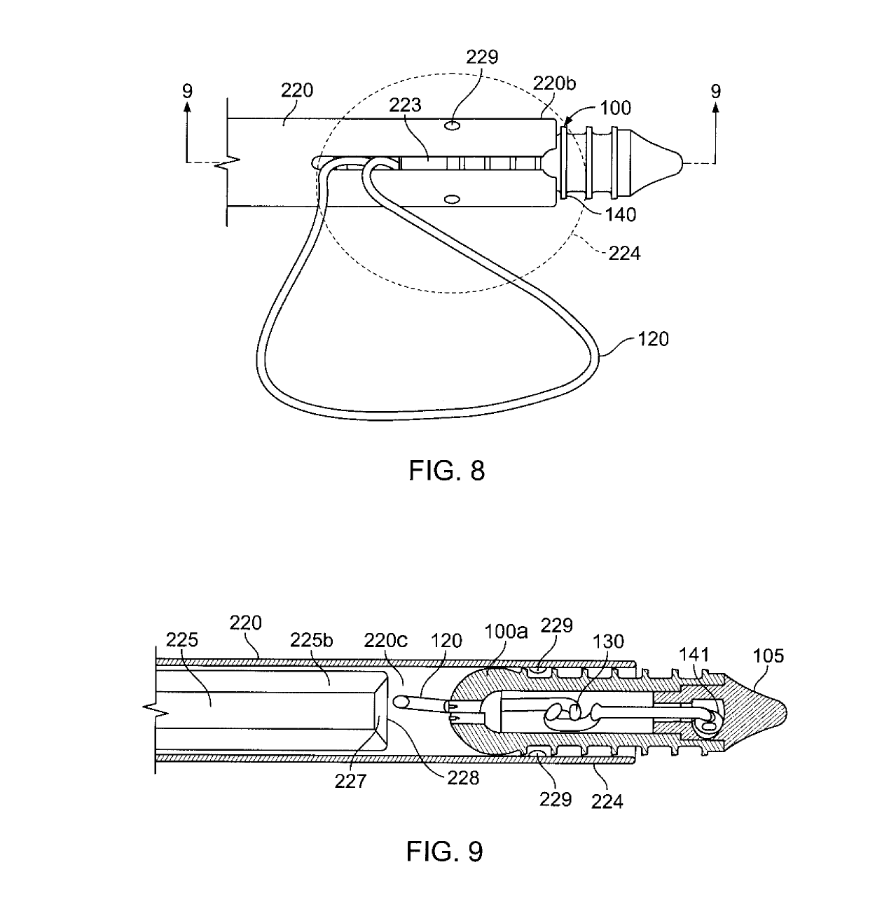

Referring to FIGS. 8 and 9, the anchor delivery device 200 defines an anchor receiving portion 224 for securing the anchor 100 in place. The driving end 228 of the distal portion 225b of the inner shaft 225 engages with the proximal portion 100a of the anchor 100. The anchor receiving portion 224 secures the anchor 100 and loop portion 120 of the suture 110 in a loaded state for delivering the anchor 100 to a hole drilled within tissue, such as the bony surface of the glenoid 12 of FIG. 1. The anchor receiving portion 224 includes raised dimples 229 formed within an interior of the lumen 220c that project inwardly and fit within the adjacent ribs 140 of the anchor when the anchor 100 is loaded into the anchor receiving portion 224. The anchor 100 is partially recessed within the outer shaft 225 while in a loaded state, e.g., with only three of the ribs 140 exposed, to maintain stability within the delivery device 200 while also ensuring enough of the anchor 100 is exposed to assist the surgeon in guiding the anchor 100 into a hole within a bony surface. The inner shaft 225 delivers the anchor 100 with an advancement force, such as by tapping with a surgical mallet, imparted through the suture puller 230. The loop portion 120 of the suture 110 is received within the slot 223 of the outer shaft 220. The recess 227 is frustoconically shaped to prevent the suture 110 from becoming pinched between the proximal portion 170 of the anchor 100 and the driving end 228 of the inner shaft 225.

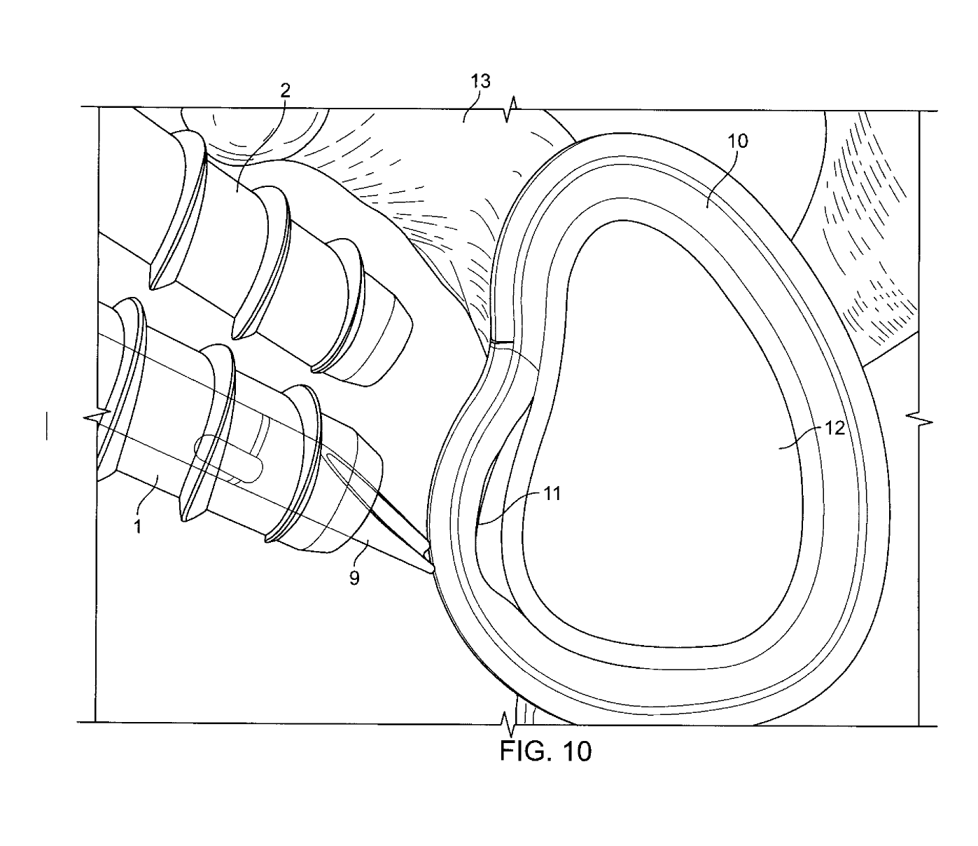

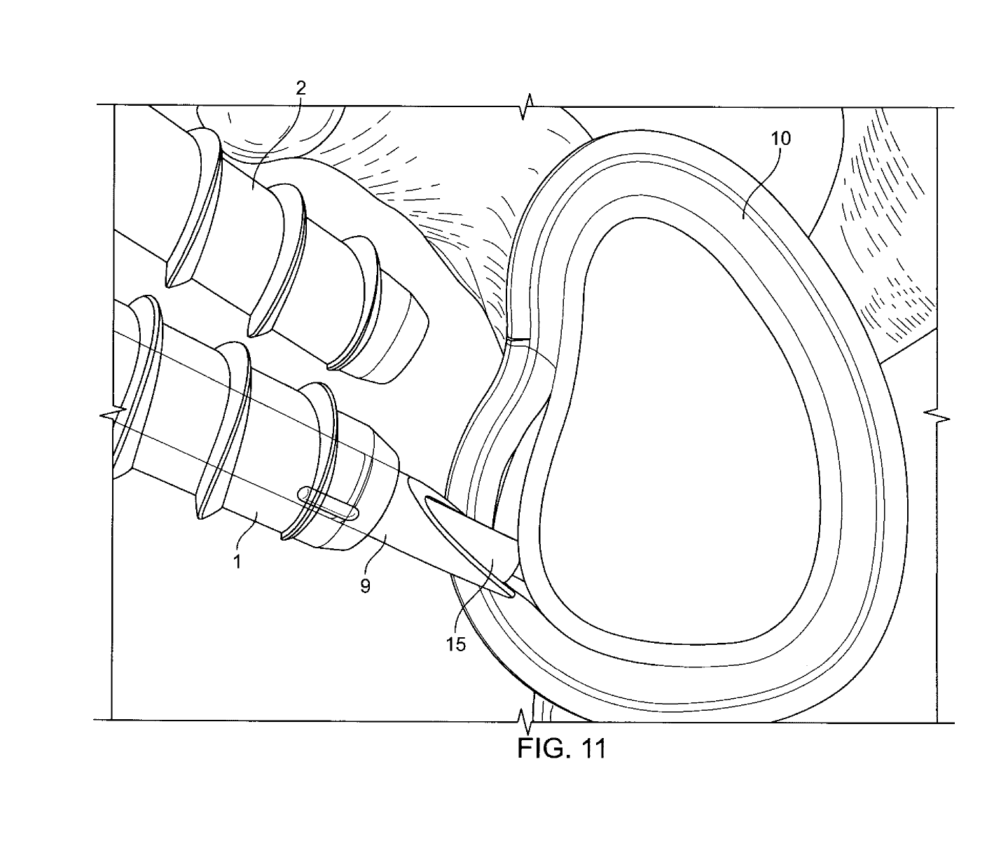

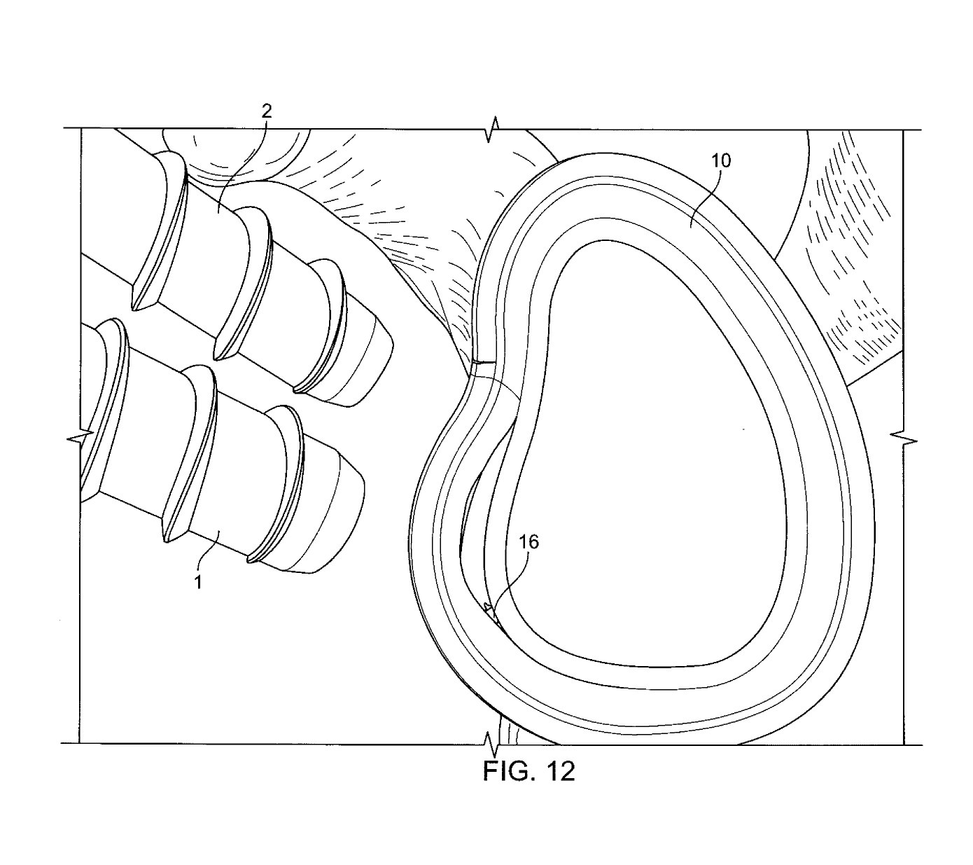

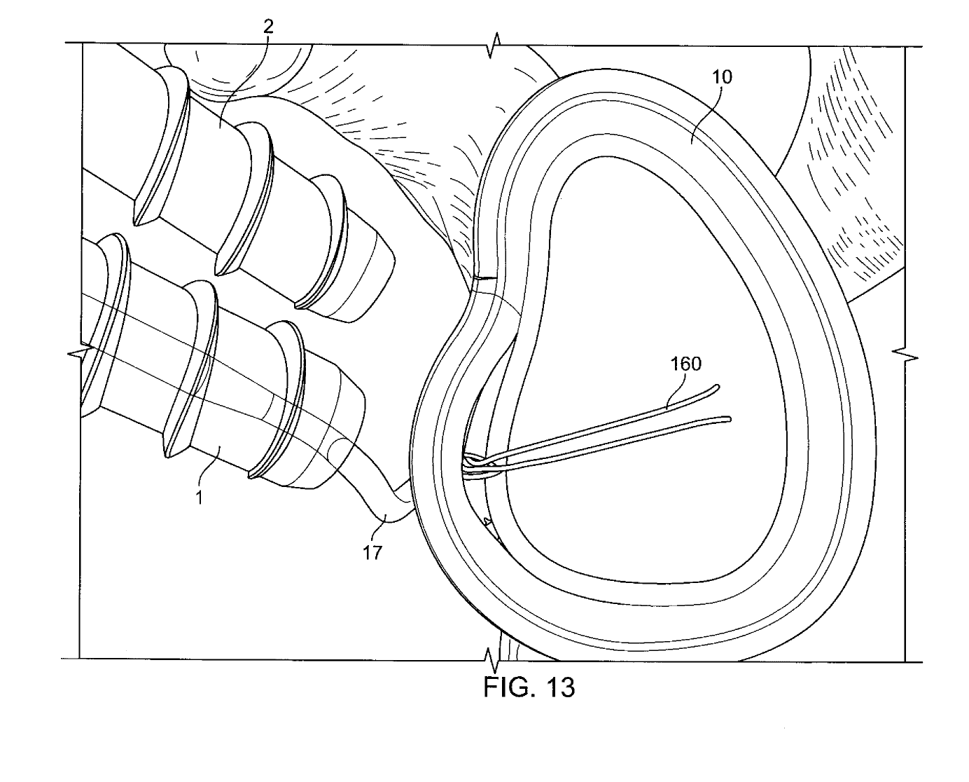

Referring to FIG. 10, in use of the surgical device 5 to repair the tear 11 in the labrum 10, a first delivery cannula 1, e.g., a superior portal, and a second delivery cannula 2, e.g., a lateral accessory portal, are positioned adjacent to the tear 11 of the labrum 10. Referring to FIGS. 11-12, a surgeon inserts a drill guide 9 through the first delivery cannula 1 to permit the advancement of a drill 15, such as a spade drill, through the drill guide 9 and into the bony surface of the glenoid 12 to form a hole 16.

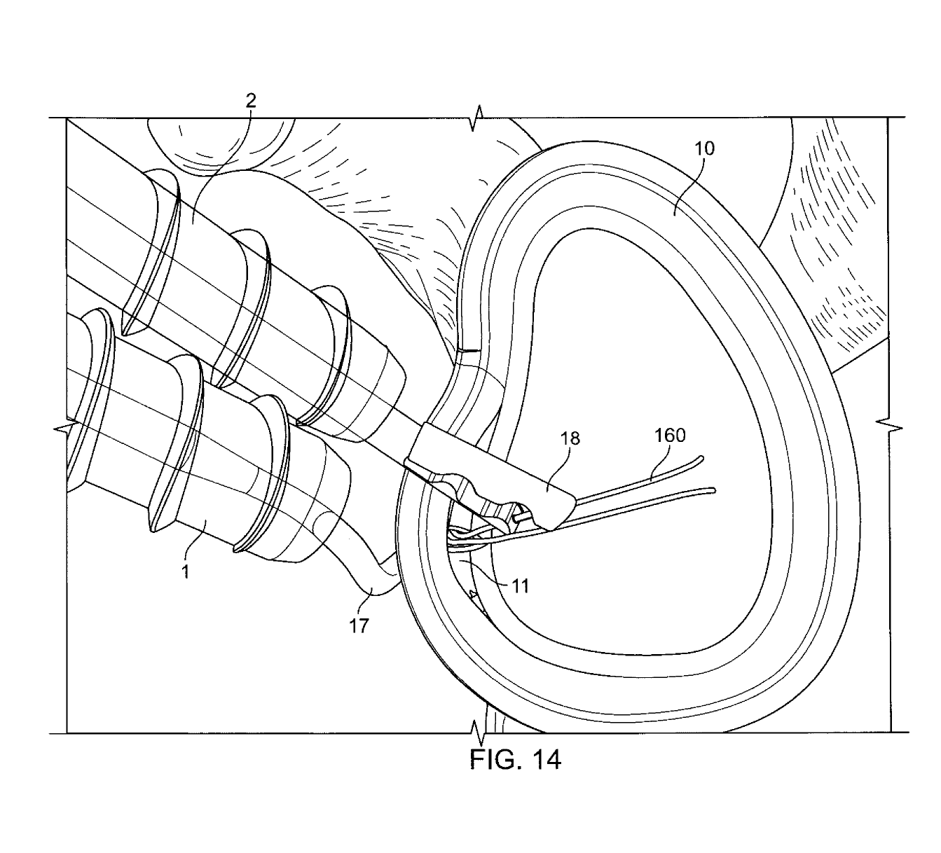

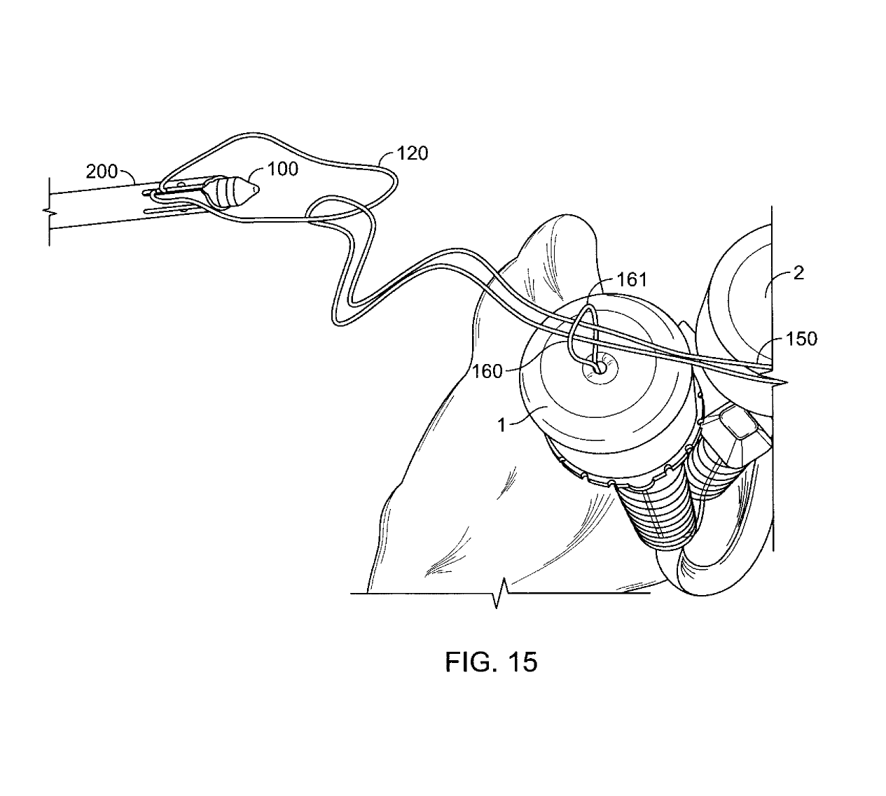

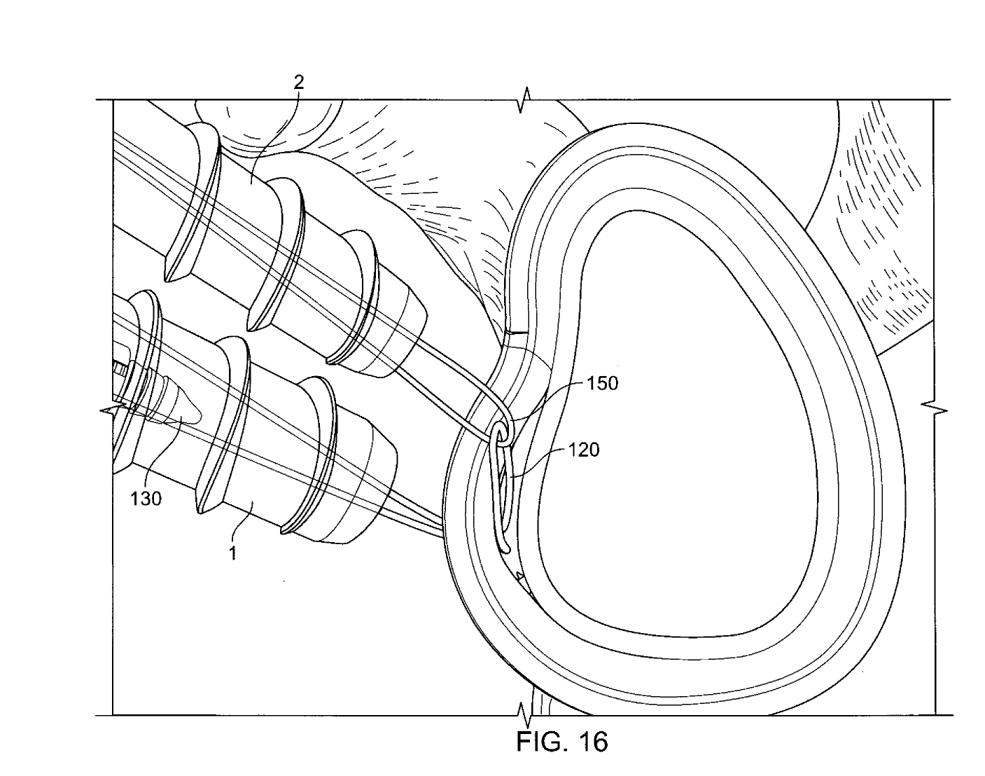

Referring to FIGS. 13-16, the surgeon inserts a suture passer 17 into the first delivery cannula 1 to advance a guiding suture 160 through the delivery portal and the tear 11 of the labrum 10. The surgeon then passes a suture retriever through the second delivery cannula 2 to retrieve free ends of the leader suture 160. The guiding suture 160 can be the same suture forming the leader or passing suture 150 of the anchor 100 or the guiding suture 160 can be a separate piece of suture (as shown in FIG. 15) having a looped portion 161 for engaging the leader suture 150 of the anchor 100. If the guiding suture 160 and leading suture 150 are separate sutures, the surgeon removes the suture retriever 17, leaving the looped portion 161 exposed at a proximal portion of the first delivery cannula 1 (see FIG. 15). The surgeon connects the leading suture 150 to the looped portion 161 of the guiding suture 160 after the suture passer 17 is removed (as shown in FIG. 15). The surgeon next advances the delivery device 200 and anchor 100 to the surgical site through a central lumen in the first delivery cannula 1 while simultaneously using the suture retriever 18 to pull the leading suture 150 through the tear 11 and outwardly through the second delivery cannula 2. The surgeon pulls the leading suture 150 outwardly through the second delivery cannula 2 until the loop portion 120 of the suture 110 passes through the tear 11, e.g., the surgeon may observe the position of the loop portion 120 arthroscopically, and into the position shown in FIG. 16.

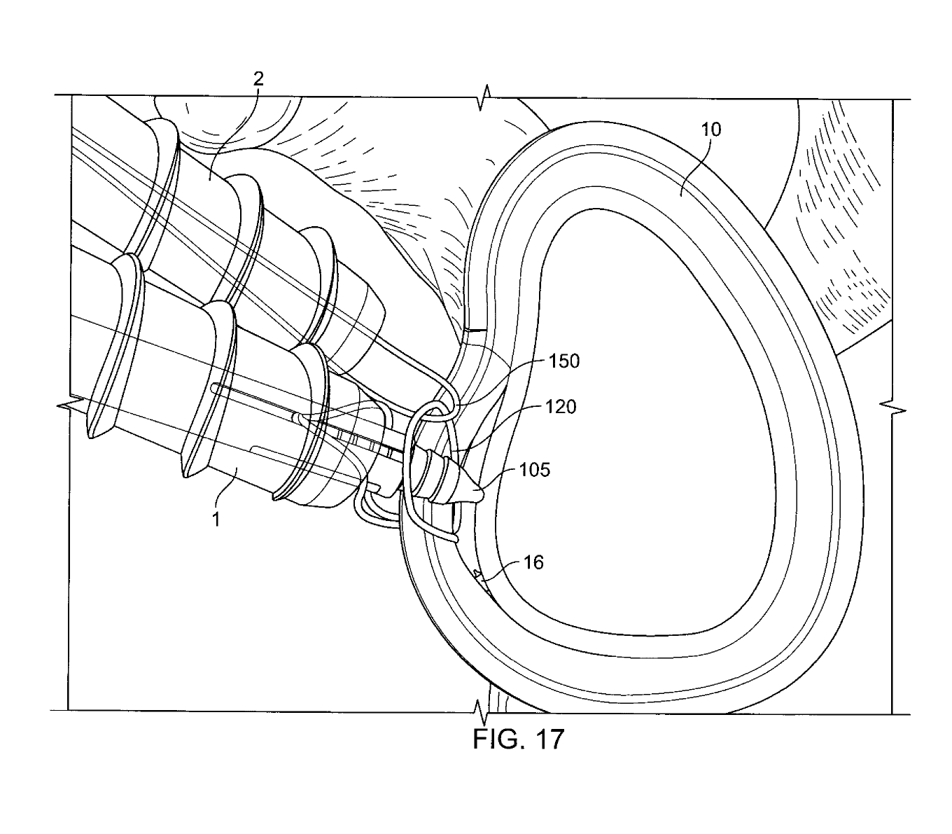

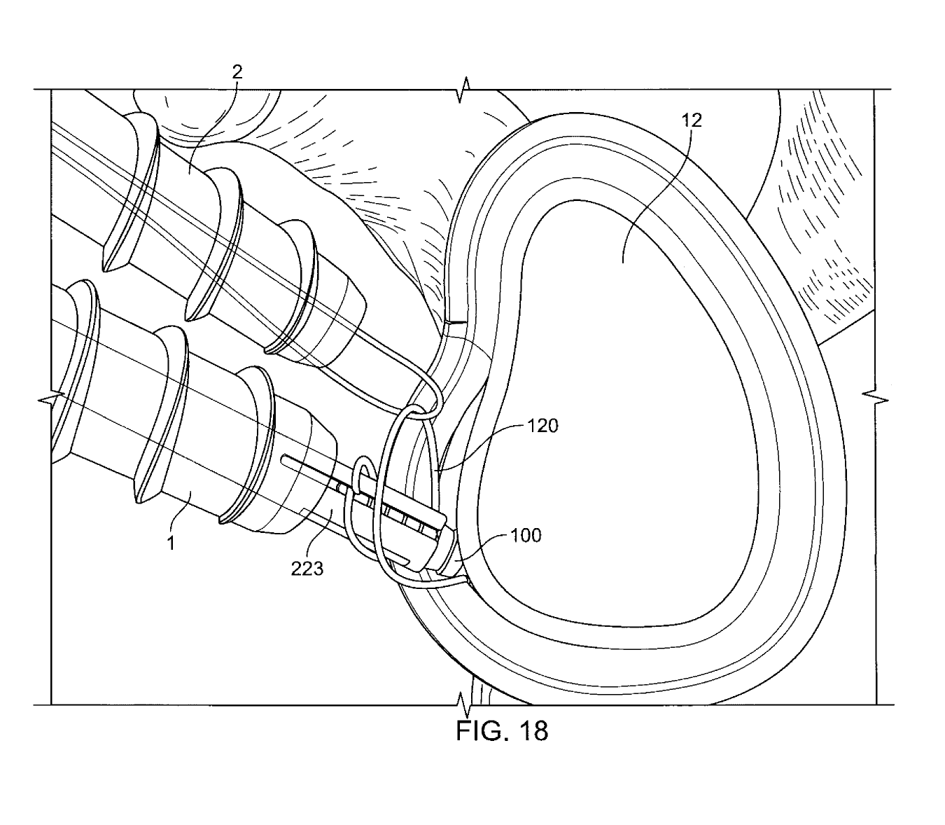

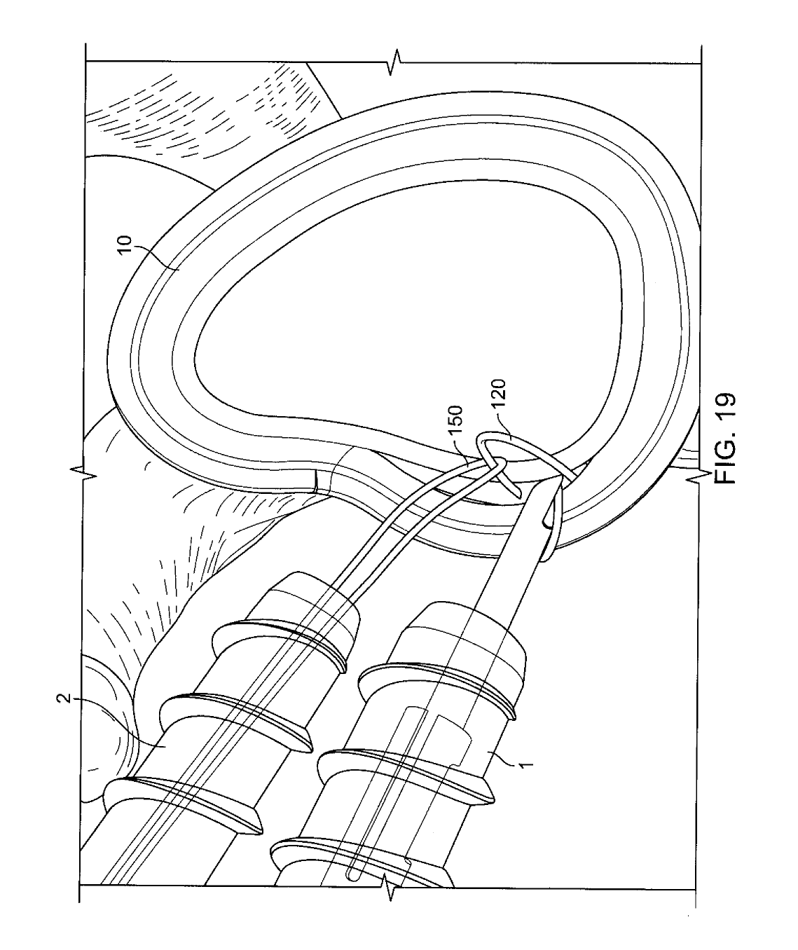

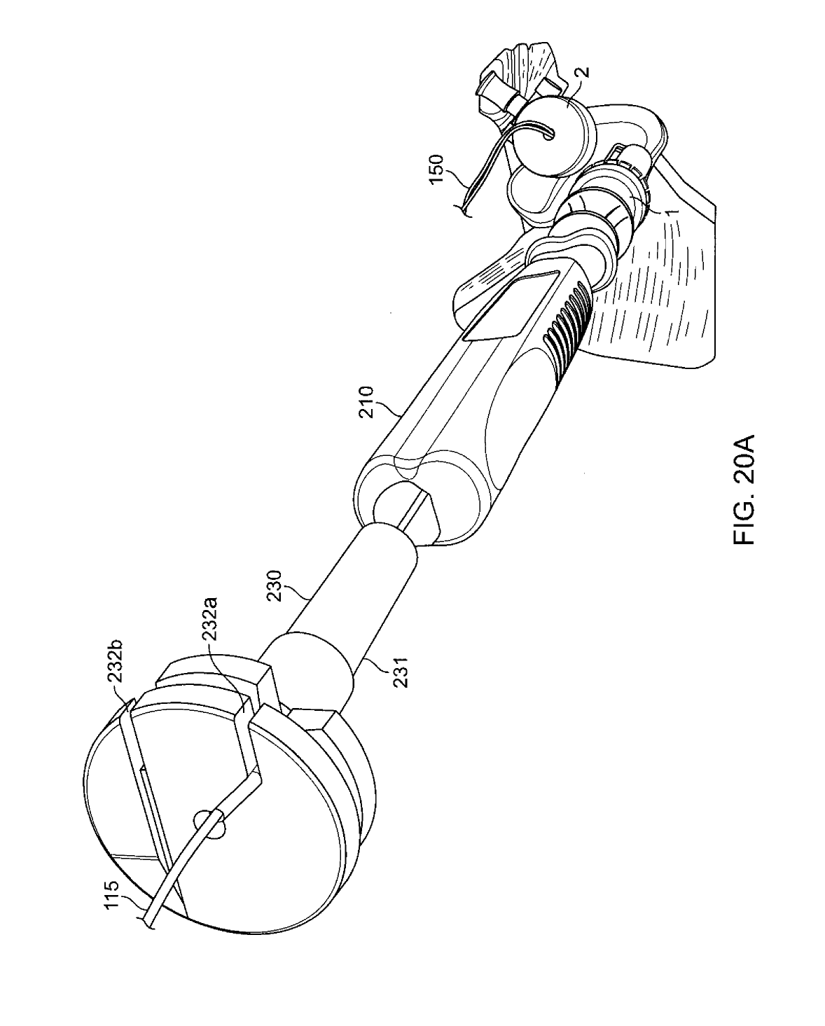

Referring to FIGS. 17-19, the surgeon passes the anchor delivery device 200 and anchor 100 through the section of the loop portion 120 that has already been passed through the tear 11. The surgeon then aligns the anchor 100 with the hole 16 and advances the anchor 100 partially into the hole 16. The surgeon imparts an advancement force, such as by tapping with a surgical mallet to the head 233 of the suture puller 230, to advance the anchor 100 into hole 16 within the bony surface of the glenoid 12. When the surgeon applies the advancement force to the head 233 of the suture puller 230, the groove 232 of the suture puller 230 maintains the tensioning portion 115 of the suture secured to the head 233 while simultaneously preventing the mallet from striking the tensioning portion 115. The surgeon pulls the tension portion 115 of the suture 110 by pulling on the suture puller 230 which has the leading end 118 secured within the groove 232. The surgeon pulls the tension portion 115 proximally to reduce the loop portion 120 and to tighten the suture 110 with respect to the labrum 10 and anchor 100, e.g., the tensioning of the suture 110 imparts a force effectively pulling the labrum 10 toward the anchor 100. The surgeon can fully tighten the suture in stages, e.g., partially advancing the anchor 100, tightening of the suture 110, advancing the anchor 100 further into the hole 16, further tightening the suture 110, etc.

Referring to FIGS. 20A-B, when the suture 110 is adequately tightened and the anchor is fully recessed within the hole 16 (20B), the surgeon removes the suture puller 230 from the handle 210. The surgeon then advances a suture retriever or other cutting instrument through the first or the second delivery cannulas 1, 2 to remove the exposed, leading end 118 of the tension portion 115 of the suture 110.

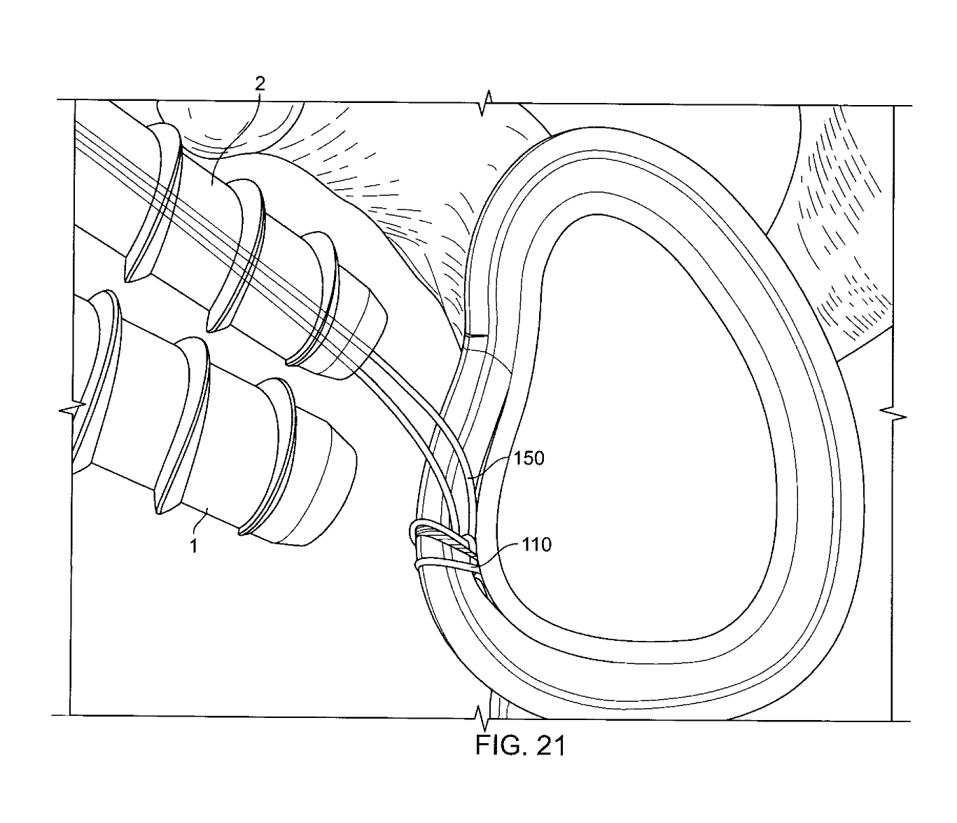

Referring to FIG. 21, once the tension portion 115 of the suture 110 has been trimmed away, the surgeon cuts the exposed portions of the leader suture 150 still remaining at the site of the repair. The foregoing procedure provides the advantages of an efficient labrum repair without the necessity of the surgeon to tie any knots at the repair site.

Referring to FIGS. 22-26, in an alternative surgical procedure, the anchor 100 is inserted into the hole 16 before the suture loop portion 120 is passed through the tissue to be repaired. Rather than repairing a torn labrum 10, as shown in FIGS. 10-21, in the alternative procedure a labrum 10 that has separated from a glenoid 12 is repaired. However, each of the procedures described with respect to FIGS. 10-21 and FIGS. 22-26 can be used to repair either a torn labrum 10 (FIG. 10) or a separated labrum (FIGS. 22-26). In addition, the alternative procedure also utilizes a first delivery cannula 1 and a second delivery cannula 2 as shown and described in connection with FIGS. 10-21.

Referring to FIG. 22, after a hole 16 has been drilled in the bony surface of glenoid 12, and prior to advancing the leader suture 150 and loop portion 120 through the tissue repair site, the surgeon inserts the anchor delivery device 200 through the first delivery cannula 1. The surgeon uses the anchor delivery device 200 to insert the anchor 100 into the hole 16 and to partially drive the anchor 100 into the hole with an axial advancement force, e.g., the tapping advancement force imparted to the suture puller 230, inner shaft 225 and proximal portion 101 of the anchor 100. The anchor 100 is recessed into the hole 16 until only the suture groove 109 of the anchor 100 is exposed.

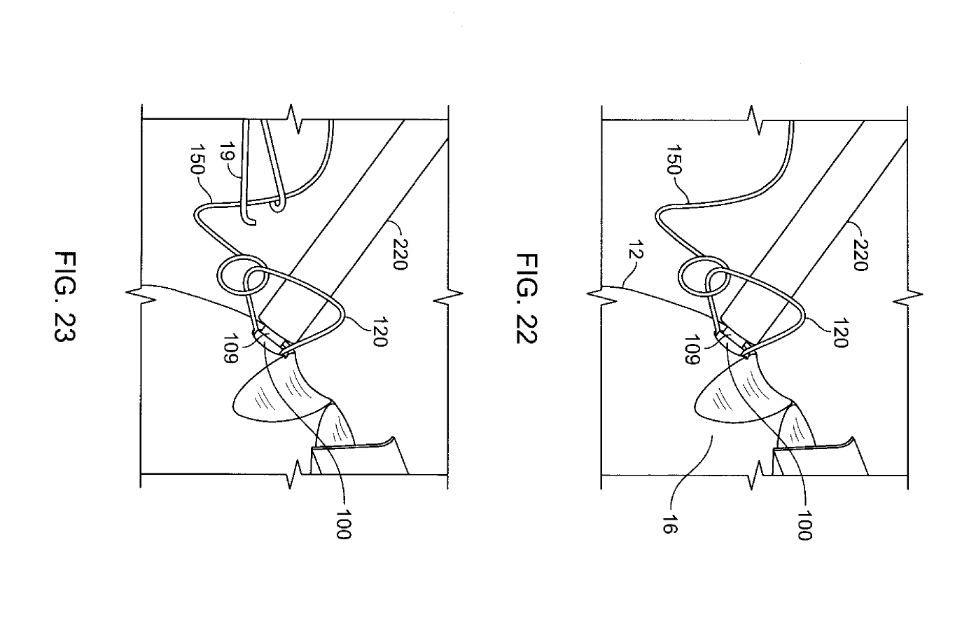

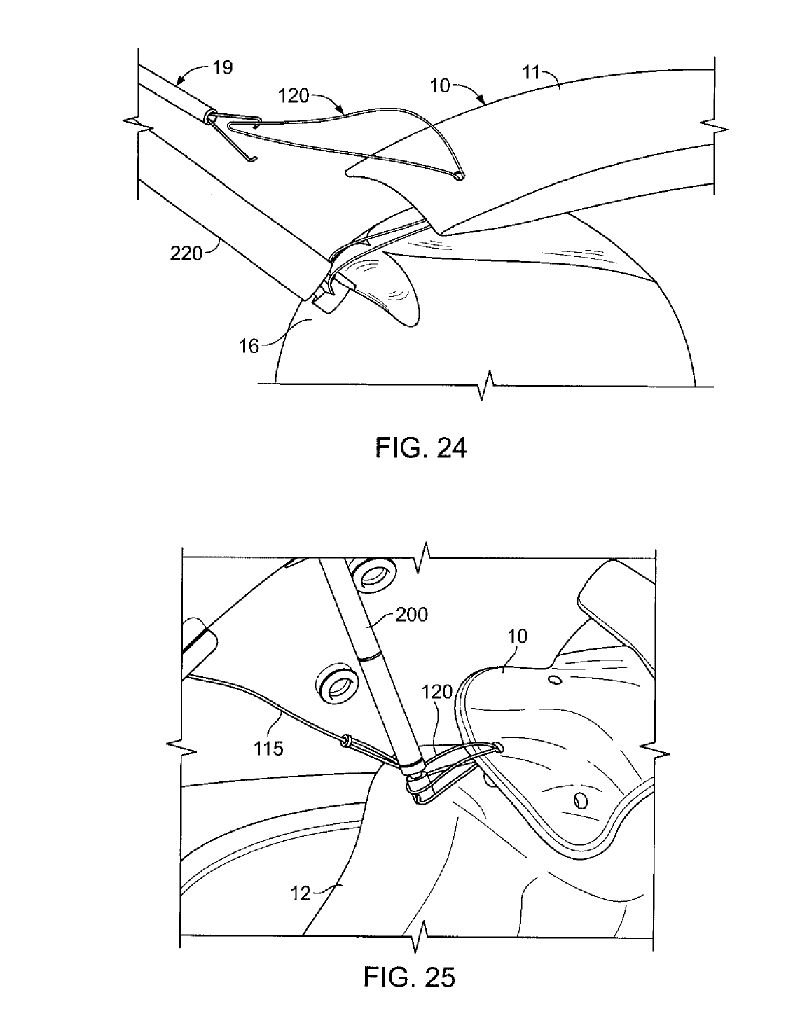

The surgeon removes the suture puller 230 and passes an open suture retriever 19 through the first delivery cannula 2 alongside the inner shaft 225 and outer shaft 220 of the anchor delivery device, and positions the suture retriever 19 adjacent to the anchor 100. The surgeon retrieves the leader suture 150 secured to the loop portion 120 with the suture retriever 19. Referring to FIGS. 23 and 24, the surgeon passes the suture retriever through the suture leader 150 and the loop portion 120 through the tissue, e.g., through a tear or hole 11 pierced in the separated labrum 10. The surgeon pulls the loop portion 120 proximally through the first delivery cannula 1 and removes the anchor delivery device 200. The surgeon repositions the loop portion 120 around the tensioning portion 115 of the suture 110 which remains in place secured to the anchor 100. The loop portion 120 is next positioned around the proximal portion 100a of the anchor 100 and brought into engagement with the suture groove 109. Alternatively, the loop portion 120 can be withdrawn from the tissue repair site after having been passed through the tissue and passed over the proximal end of the anchor delivery device 200. The loop portion 120 is then passed down along the delivery device 200, e.g., without removing the anchor delivery device 200, and into in engagement with the suture groove 109.

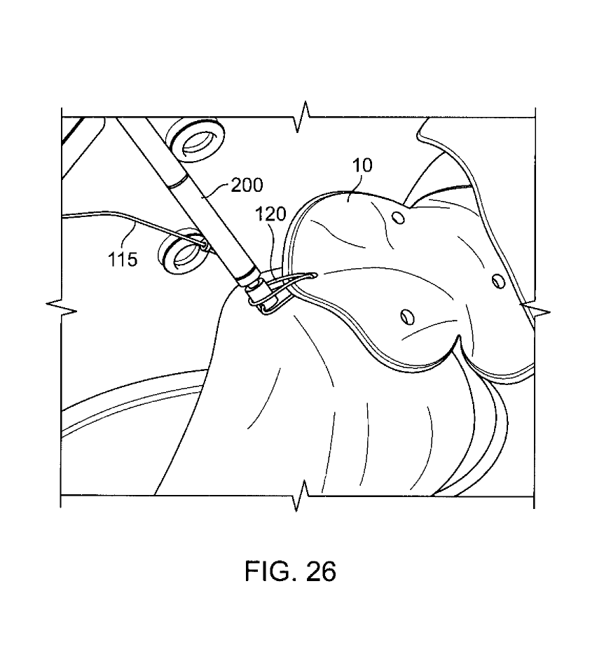

Referring to FIGS. 25-26, the surgeon reinserts the delivery device 200 to hold the proximal portion 100a of the anchor 100 in place. The surgeon pulls on the tension portion 115 with the suture retriever 19, or by hand if tension portion 115 is drawn through the first delivery cannula 1, to reduce the loop portion 120 and pull the labrum 10 toward the anchor 100. The suture groove 109 effectively serves as a pulley that permits the suture loop portion 120 to slide therein and to be tensioned responsive to the pulling of the surgeon. After the loop portion 120 has been reduced, the suture 110 is tightened and the labrum 10 is pulled into a repaired state, the surgeon uses the delivery device 200 to fully recess the anchor 100 into the hole 16 below the bony surface 14 of the glenoid 12. The delivery device 200 is then removed and the exposed ends of the leader suture 150 or tension portion 115 are trimmed by the surgeon.

As discussed in connection with the procedure of FIGS. 10-21, the procedure described in connection with FIGS. 22-26 provides the advantages of an efficient labrum repair without the surgeon having to tie any knots at the repair site. In addition, the procedure described in connection with FIGS. 22-26 is suitable for repairing the torn labrum 10 shown in FIG. 10.

Other embodiments are within the scope of the following claims.

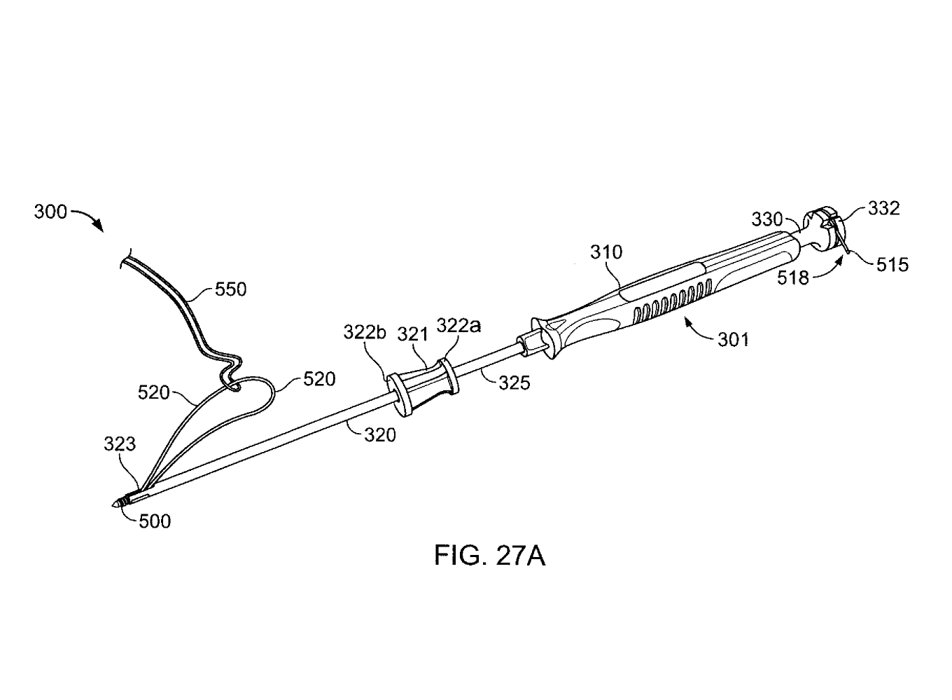

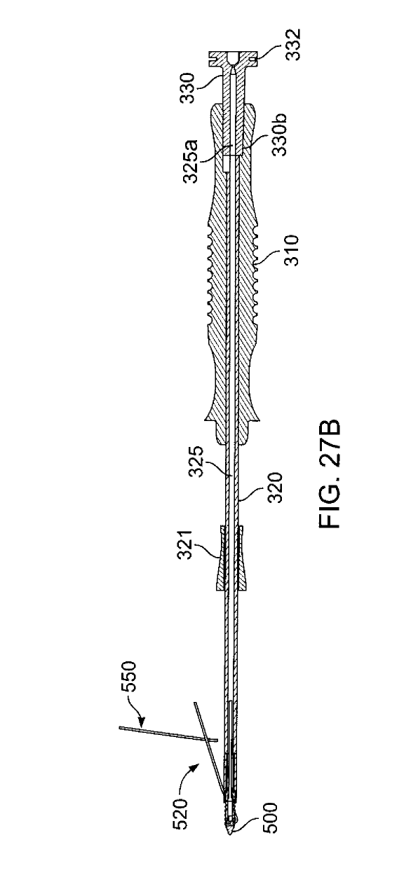

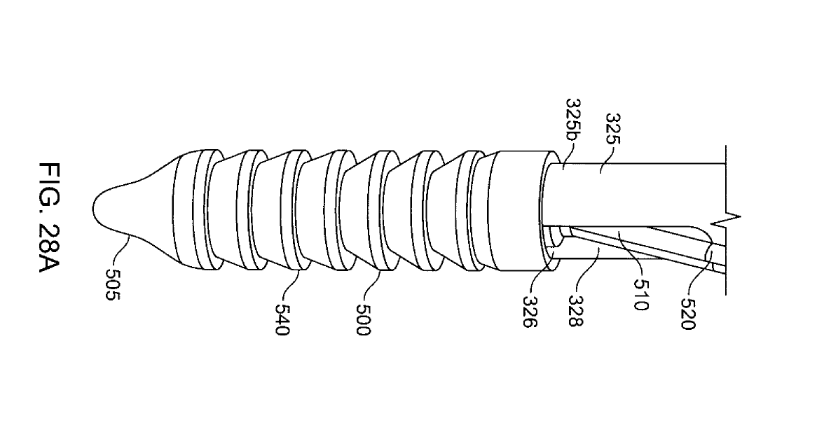

For example, referring to FIGS. 27A-29, an alternative surgical instrument 300 includes an anchor delivery device 301 and anchor 500. The anchor delivery device 300 includes a handle 310, a suture puller 330 and an inner shaft 325 received within the handle 310, as described above with respect to the previous embodiment. Rather than a fixed outer shaft, delivery device 300 includes a floating outer shaft 320 that is not secured to the handle 310 and is permitted to move axially relative to the inner shaft 325 and handle 310. The outer shaft 320 includes a depth stop 321 at its proximal end 320a having opposed flanges 322A, 322B that restrict movement of the outer shaft in a proximal direction by the flange 322A abutting against the handle 310, and in a distal direction by the flange 322B abutting against the proximal end of a delivery cannula 1. In this way, any tendency for the advancement force imparted to the suture puller 330 and inner shaft 325 to be applied to the outer shaft 320 is limited. The inner shaft 325 can be provided with a keyway, e.g., similar to the longitudinally extending flat portion 226 which engages a corresponding surface within the handle 310.

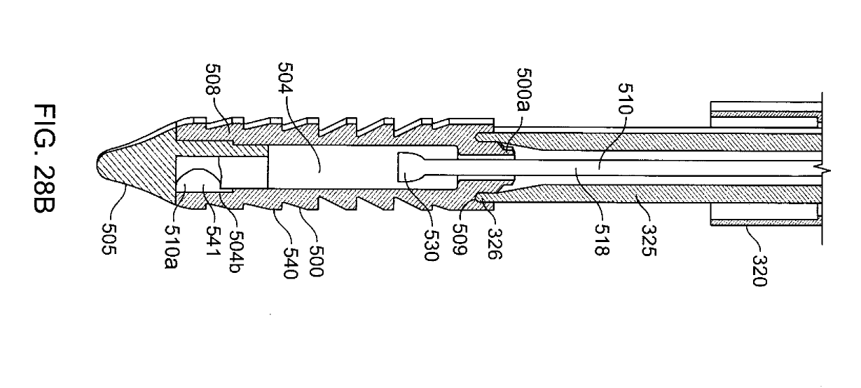

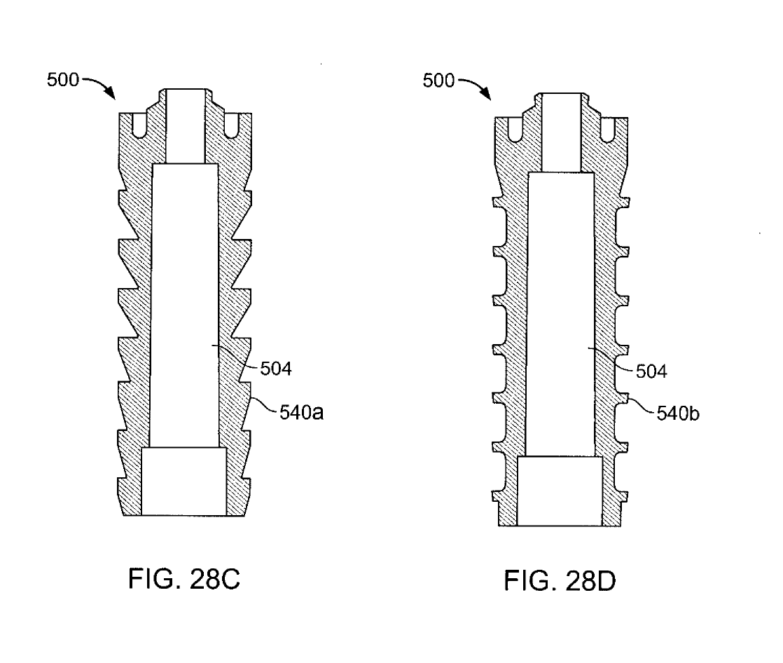

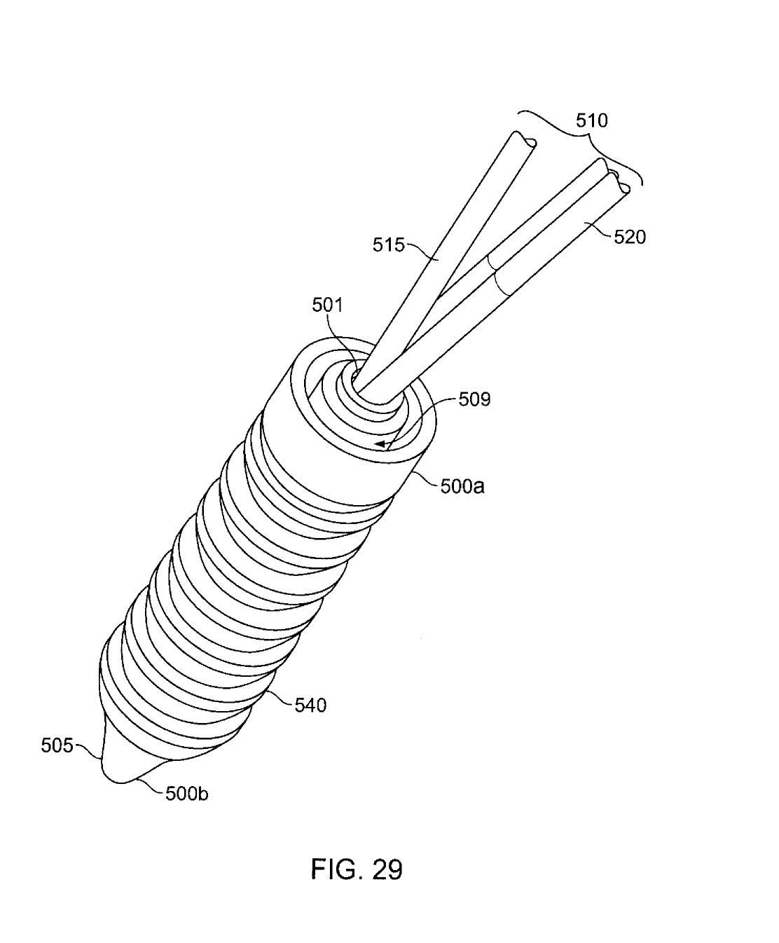

Referring to FIG. 27B, the inner shaft 325 includes a proximal portion 325a and the suture puller 330 includes a distal portion 330b. The distal portion 330b of the suture puller 330 couples with and receives the proximal portion 325a of the inner shaft 325 with a male-female fit. Alternatively, the suture puller 330 and inner shaft 325 can abut against each other without the male-female fit shown. Referring to FIGS. 28 and 29, the anchor 500 includes a proximal face 500a coupled with a suture 510 having a tensioning portion 515 and a loop portion 520. As described in connection with the suture puller 230 of FIG. 2, the suture puller 330 includes a circumferentially extending groove 332 for accommodating the leading end 518 of a suture 510.

The anchor 500 includes an anchor body 508 and an anchor plug 505. The anchor body 508 includes an anchor cavity 504 and ribs 540, and the proximal face 500a of the anchor 500 has a circumferential drive recess 509 formed therein. Rather than including a plurality of suture holes, the anchor 500 includes a single, centrally oriented and cylindrically shaped suture hole 501 permitting the suture loop portion 120 and tensioning portion 115 to extend from the anchor cavity 504. The anchor cavity 504 is a longitudinally extending, hollow cavity having a generally cylindrical shape and a stepped portion at a distal portion 504b of the anchor cavity 504 which forms a recess for the anchor plug 505.

Referring to FIGS. 28B, the suture 510 includes a sliding knot 530 tied and secured exterior to the cavity 504 and drawn inwardly into anchor cavity 504 after the sliding knot 530 is tied. A first end 510a of the suture 510 is passed through a hole 541 in the plug 505 and is secured after the plug 505 is press-fit into the distal portion 504b of the anchor cavity 504. The anchor body 508 includes circumferentially extending ribs 540 that secure the anchor 500 within tissue after being inserted, e.g., such as by tapping with a surgical mallet, into a target tissue. Referring to FIGS. 28B-C, the ribs 540 can include stacked, truncated conically shaped ribs 540a. Referring to FIG. 28D, alternatively, or in combination, the ribs 540 can include circumferentially extending and uniform disc-shaped ribs 540b.

Rather than abutting against the anchor 500, the distal end 325b of the inner shaft 325 of the anchor delivery device 300 includes a corresponding drive portion 326 that is received within the drive recess 509 formed in the anchor 500. During delivery of the anchor 500, the inner shaft 325 is maintained in engagement and axially aligned with the anchor 500 through the drive recess 509 and drive portion 326. The inner shaft 325 includes a longitudinally extending slot 328 or recess through which the suture loop portion 520 extends laterally from the inner shaft 325. The inner shaft 325 includes a lumen 325c receiving the tensioning portion 515 which extends through the lumen 325c and through the suture puller 330. The outer shaft 320 includes a longitudinally extending slot 323 which also permits the suture tensioning portion 515 and loop portion 520 to extend from the proximal face 500a of the anchor 500, through the slot 328 and outwardly through the slot 323. Although the anchor delivery device uses a floating outer shaft 320, the anchor delivery device 300 is otherwise used in the same manner as the delivery device 200 described above.

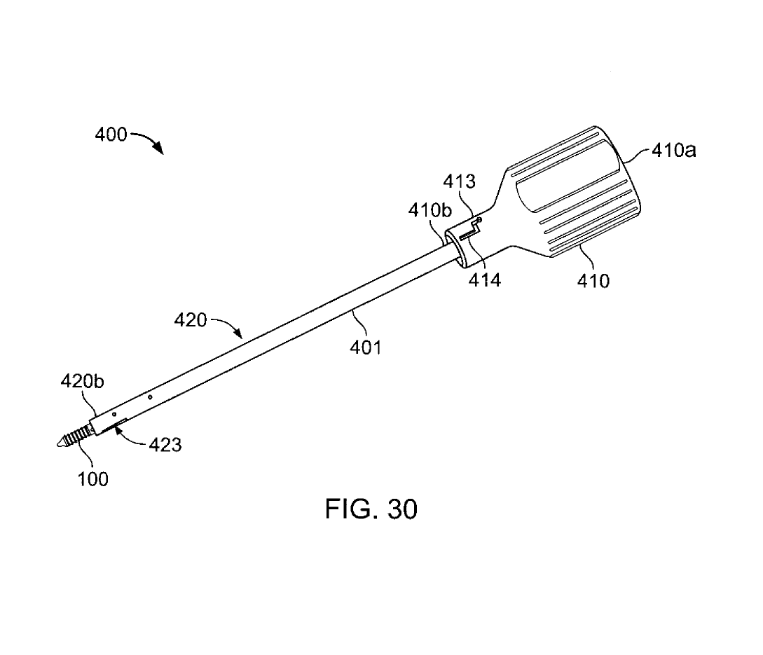

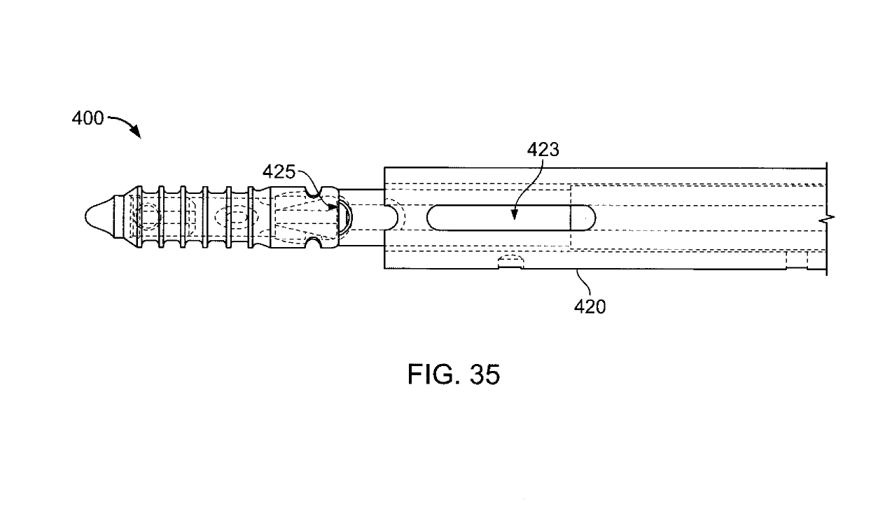

Referring to FIGS. 30-35, an alternative surgical instrument 400 includes an anchor delivery device 401 and an anchor 100, e.g., any of the anchors described herein. Delivery device 401 includes an outer shaft 420, an inner shaft 425 and a handle 410. The outer shaft 420 is a retractable and rotatable sheath that is movable with respect to both the inner shaft 425 and the handle 410, as described below. The outer shaft 420 is retractable and rotatable to preset positions which ensure the anchor 100 is inserted to a predetermined anchor insertion depth. Anchor 100 is loaded and held within the retractable outer shaft 420. The inner shaft 425 is coupled with the handle 410, and the outer shaft 420 is indirectly coupled to the handle 410 through an outer shaft hub 411. Rather than having a movable inner shaft, the inner shaft 425 is fixed with respect to the handle 410. The handle 410 includes a proximal cavity 410a for receiving a flexible seal 417 and a distal cavity 410b for coupling with the outer shaft hub 411.

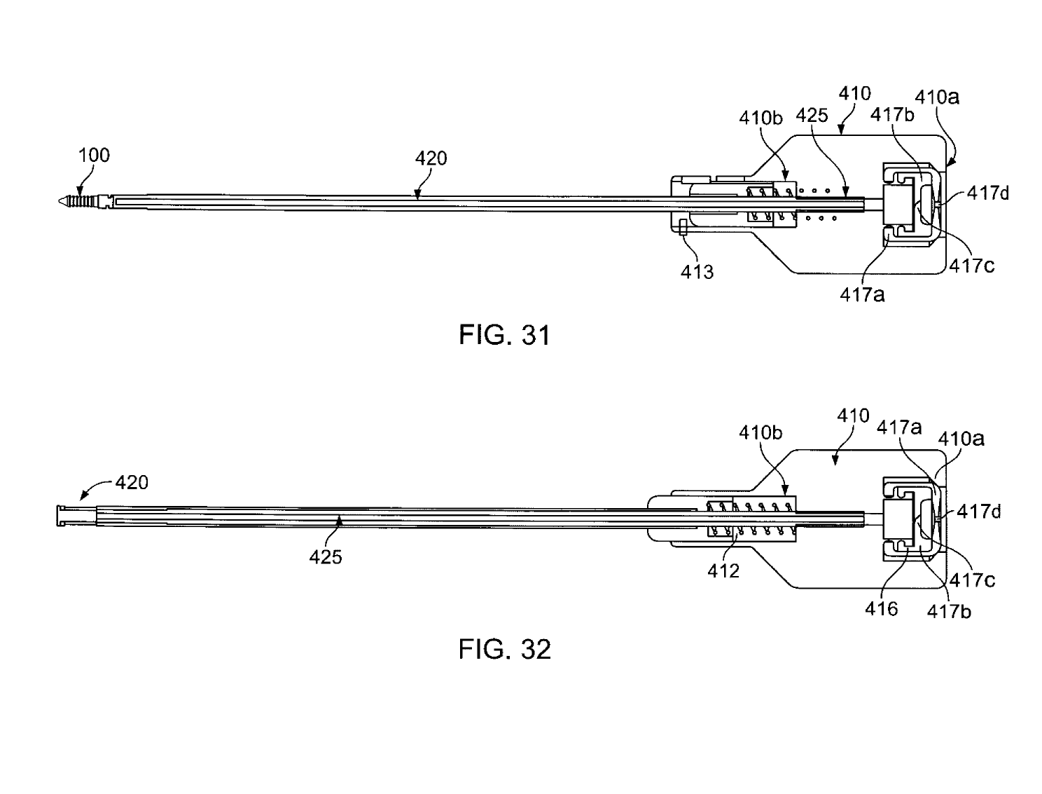

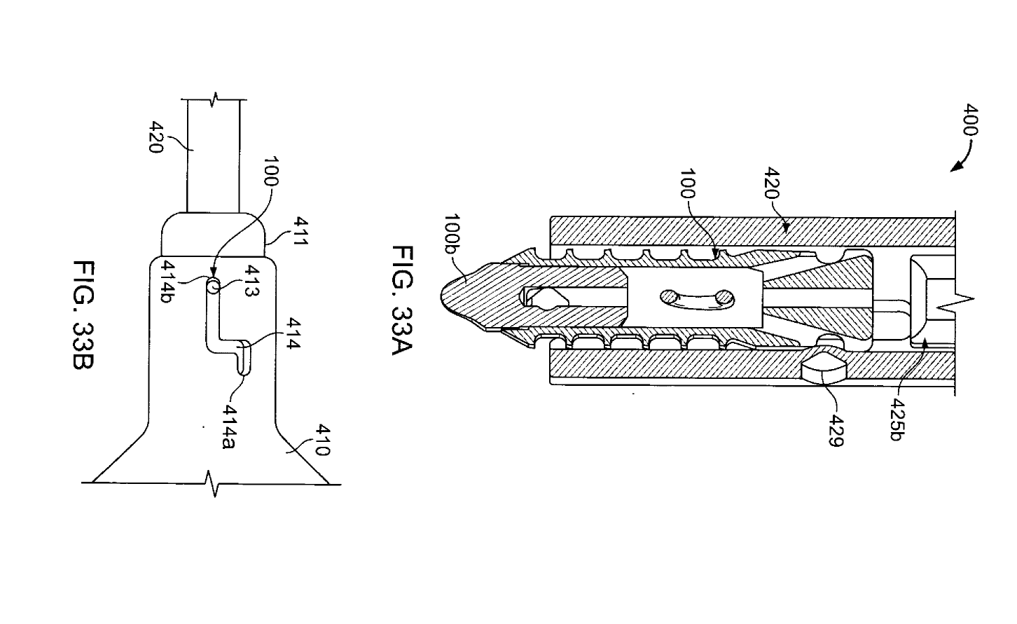

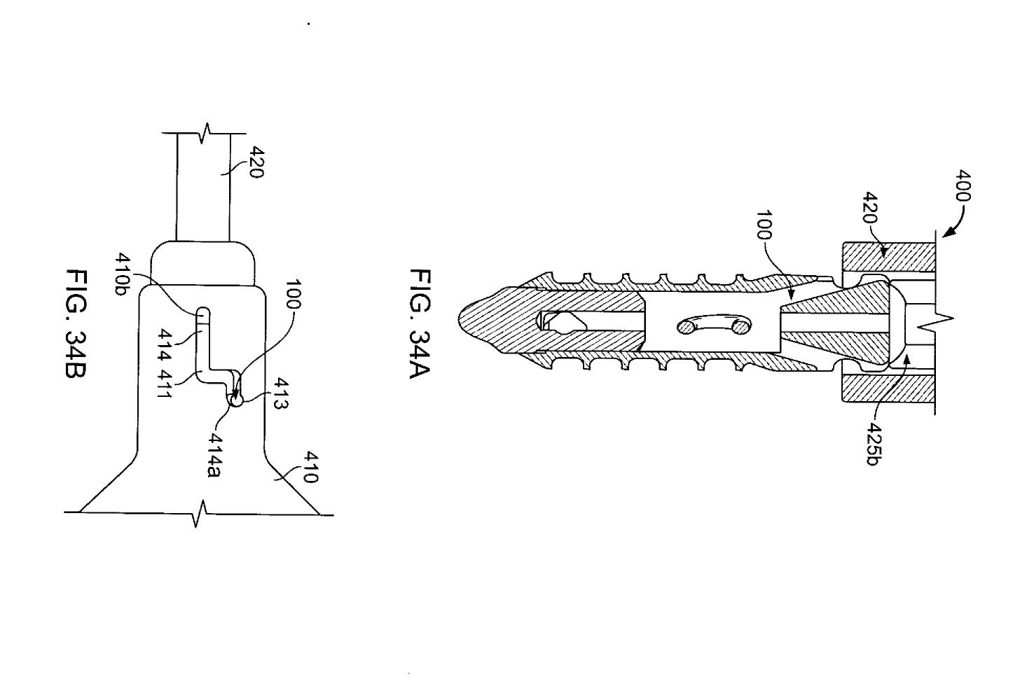

The outer shaft hub 411 is a cylindrical tube fitted within the distal cavity 410b of the handle 410. The outer shaft hub 411 permits both axial and rotary movement of the outer shaft 420 relative to the handle 410, e.g., an inner portion 411a of the outer shaft hub 411 acts as a bearing surface for the rotatable outer shaft 420. The outer shaft hub 411 includes a locator pin 413 which extends outwardly from the hub 411 and is received within a locator slot 414 defined by an exterior of the handle 410 for controlling the axial and rotary movement of the hub 411 and outer shaft 420 between an axially extended position (FIGS. 32, 33A, 33B) and a circumferentially offset and retracted position (FIGS. 31, 34A, 34B). A spring 412 is provided between the outer shaft hub 411 and a proximal end of the distal cavity 410b. The spring 412 provides a torsion and compression force which biases the outer shaft hub 411 when it is rotated and retracted within the distal cavity 410b. The outer shaft 420 passes through a lumen 411c of the outer shaft hub and is press fit within the lumen 411c. The spring 412 provides a spring force permitting offsetting rotary and/or axial movement of the hub 411 when the outer shaft 420 is retracted and rotated into the retracted position (FIGS. 31, 34A, 34B).

The proximal cavity 410a of the handle 410 also includes the optional flexible seal 417. The flexible seal 417 includes a pair of opposed, flexible diaphragms 417a, 417b that each define opposing, central through holes 417d, 417c, respectively. The through holes 417c, 417d permit the insertion of additional devices through the handle 410, e.g., such as a suture retriever or suture puller for imparting a force by hand-pressure, by tapping with a surgical mallet, or other axially oriented, advancement force to the inner shaft 425 through the handle 410. In addition, the through holes 417c, 417d accommodates the tension portion 115 of the suture 110 of the anchor 100. The flexible seal 417 minimizes water/fluid leakage through the through holes 417c, 417d during a procedure, e.g., such as while inserting and/or removing various instruments from the through hole 417.

Referring to FIGS. 30 and 33-35, the retraction and rotation of the hub 411 and outer shaft 420 is controlled with the locator pin 413 secured to or formed with the hub 411, and the corresponding locator slot 414 formed within the handle 410. The locator slot 414 serves as a track for the locator pin 413 to limit retraction and rotation of the outer shaft 420. The locator pin 413 is connected to the hub 411, but can be formed integrally with the hub 411 and/or outer shaft 420.

In a first position with the locator pin positioned at the distal end 414b of the slot (FIGS. 33A-B), the anchor 100 is held within a distal portion 420b of the outer shaft 420 by the walls of a lumen 420c of the outer shaft. The outer shaft 420 can include one or more protrusions 429 extending inwardly from the walls of the lumen 420c, such as the dimples 229, to secure the anchor 100 within the lumen 420c. A distal end 425b of the inner shaft 425 presses against the anchor 100 to impart an advancement force against the anchor 100. In the first position, only a distal portion 105 of the anchor 100 is exposed, e.g., not covered by the outer shaft 420. The outer shaft 420 covers a majority of the anchor 100 to protect the anchor 100, as well as maintains the anchor 100 in a proper axial orientation as the anchor 100 is delivered into a bone hole. As the anchor 100 is advanced, e.g., with a tapping advancement force applied to the inner shaft 225 through a suture puller inserted through the through holes 417c, 417d or by a surgeon using hand pressure to urge the anchor delivery device 400 against bone, the outer shaft 420 retracts and rotates against the biasing force of the spring 412 until the locator pin 413 comes to rest at a second position with the locator pin positioned at the proximal end 414a of the slot (FIGS. 34A-B). Accordingly, the locator slot 414 serves as a depth stop that prevents the insertion of the anchor beyond a predetermined depth, e.g., corresponding to the overall axial length of the locator slot 414.

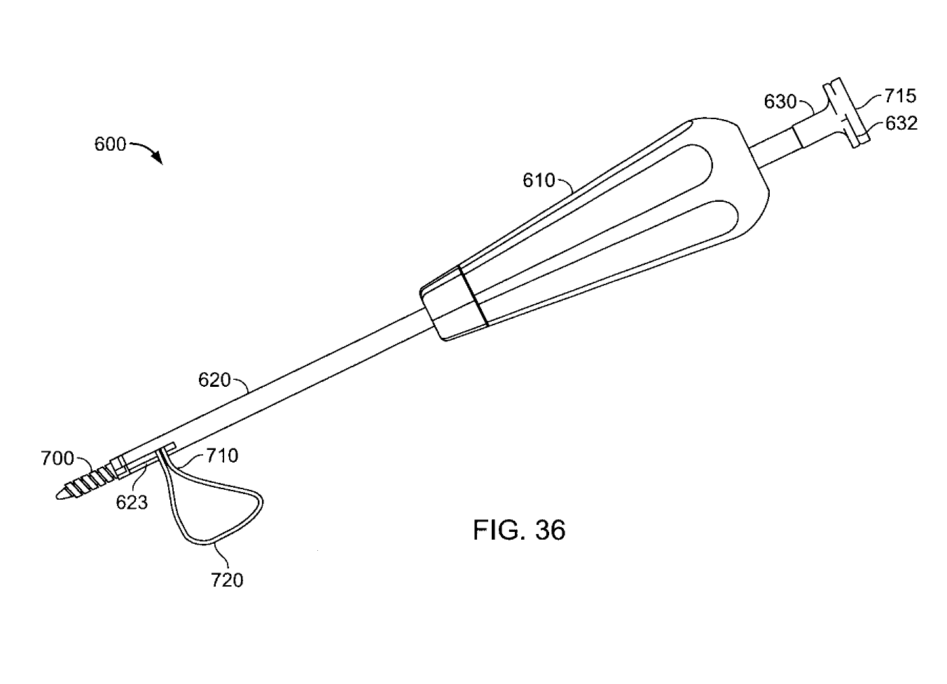

In the second position (FIGS. 34A-B), the anchor 100 is nearly driven flush with or is slightly recessed, e.g., if a suture groove 109 is provided on the anchor 100, with respect to a bony surface. The suture loop portion 120 is exposed from the anchor delivery device 400 through a longitudinally extending slot 423 (FIG. 36) formed at a distal end # of the outer shaft 420. The inner shaft 425 includes a slot or recess 425d that limits the suture loop portion 120 from being pinched between the inner shaft 425 and movable outer shaft 420 which retracts as the anchor is driven into bone (FIG. 36). The loop portion 120 of the anchor 100 is remains exposed after the anchor 100 is inserted and the anchor delivery device 400 is removed.

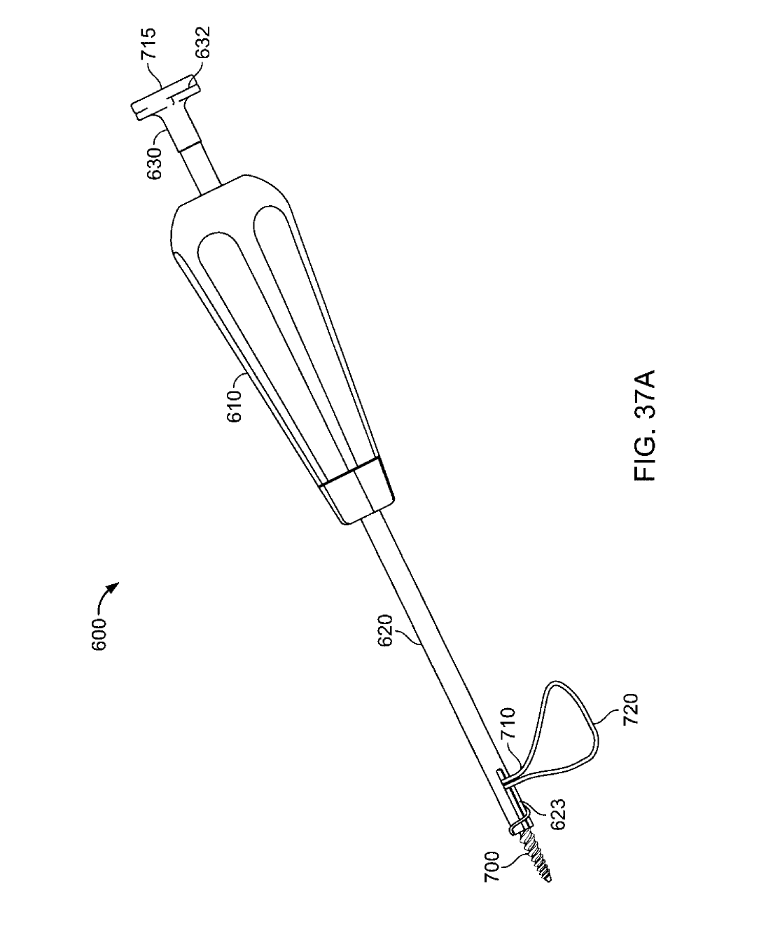

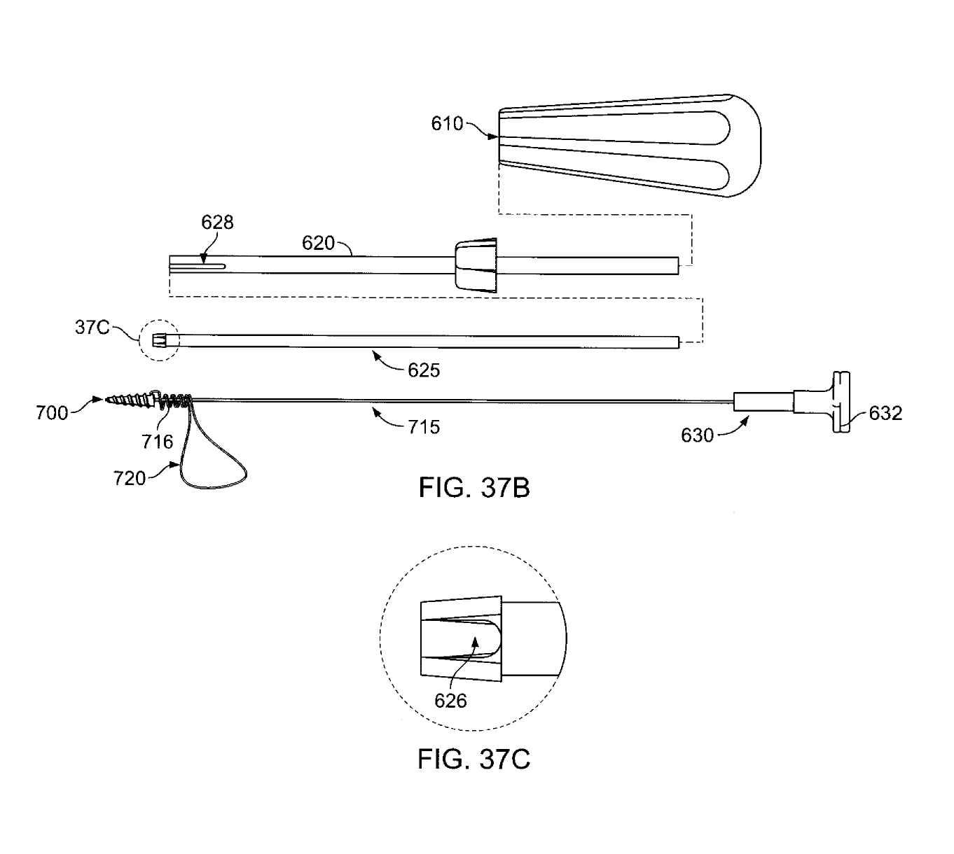

Referring to FIGS. 37A-39, an alternative surgical instrument 600 includes an anchor delivery device 601 and an anchor 700 that is advanced into a bone hole by rotation of the anchor delivery device 601 and anchor 700. The anchor delivery device 601 includes a handle 610, a movable inner shaft 625, a stationary outer shaft 620, and a suture puller 630. The anchor 700 is rotated by rotating the handle 610 and the inner shaft 625 which is connected to the handle 610.

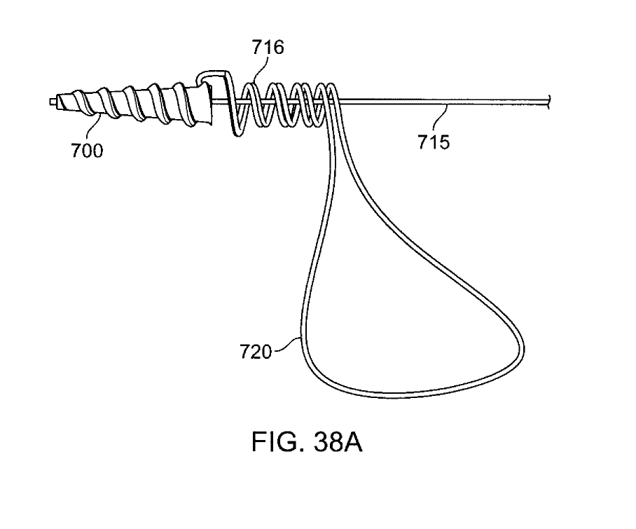

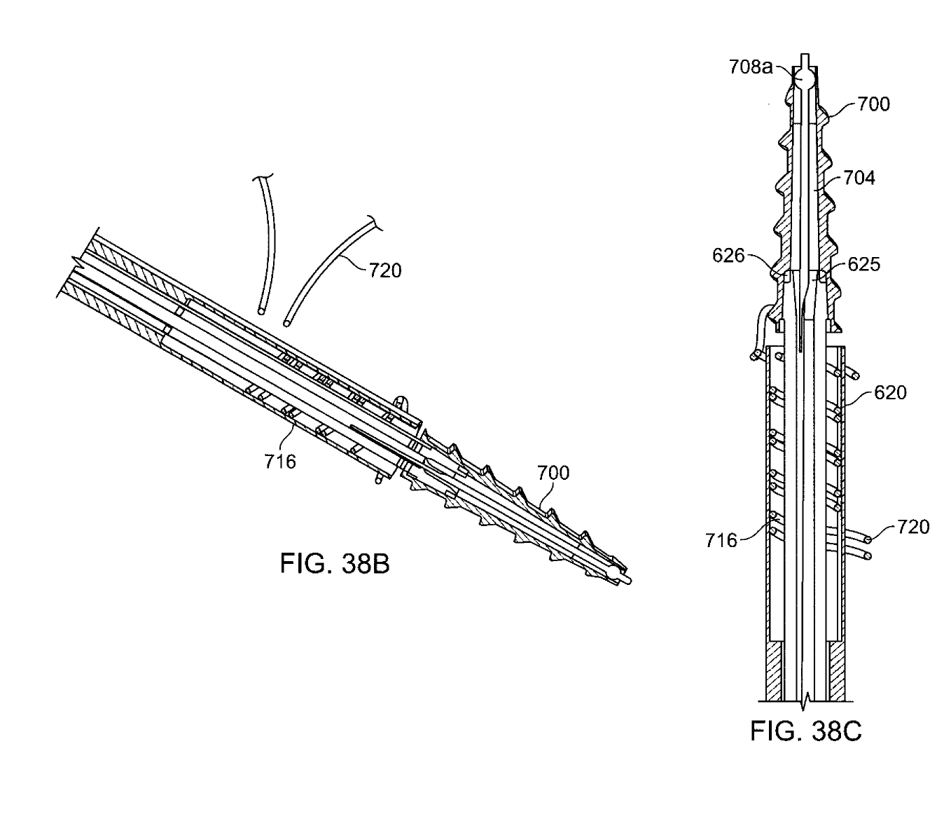

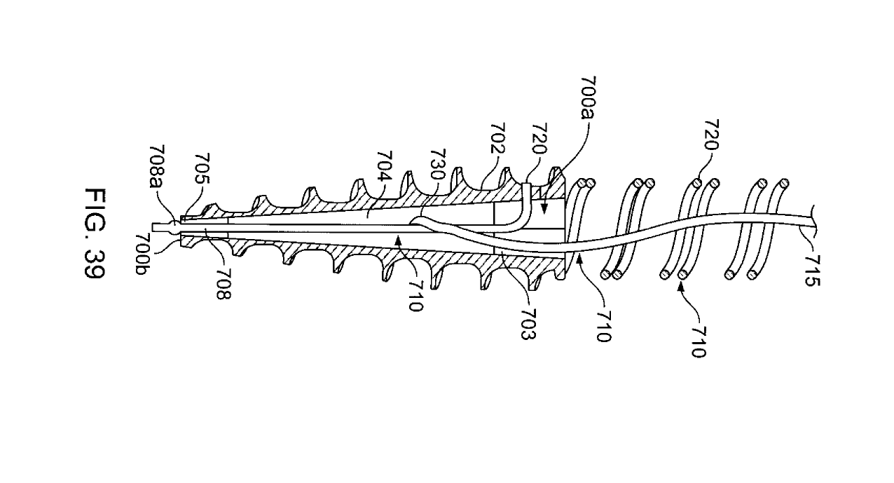

Referring to FIGS. 37A-37B and 38B-38C, the inner shaft 625 includes an anchor drive portion 626 for engaging a proximal portion 700a of the anchor 700, e.g., the drive portion 626 is formed in a distal portion 625b of the inner shaft 625, for imparting a rotational force from the inner shaft 625 to the anchor 700. The stationary outer shaft 620 includes an anchor receiving portion 624 defined by a distal end 620b of the outer shaft 620. The anchor 700 is held in a stable delivery position within the anchor receiving portion 624 of the outer shaft and is engaged with the drive portion 626 of the inner shaft 625 during delivery. The drive portion 626 can include a contoured drive surface for engaging a corresponding surface on a proximal portion of the anchor 700, e.g., hexagonally- or octagonally-shaped socket drive and driven surfaces.

The proximal portion 700a of the anchor 700 defines a circumferentially extending drive recess 626 which receives the drive portion 626 of the inner shaft 625 with a male-female fit. The anchor 700 includes a suture 710 having a loop portion 720 and a tensioning portion 715. The tensioning portion 715 extends through the suture puller 630 and is secured within a groove 632 defined by a head 630a of the suture puller. The loop portion 720 includes a wrapped portion 716 that is wrapped around inner shaft 625 and/or the tension portion 715 in a direction opposite to the rotational direction imparted to the anchor 700 during delivery. The inner shaft 625 is provided with a slot or recess 626 and the outer shaft 620 is provided with a longitudinally extending slot 623 for receiving the loop portion 720. The combination of the recess 626 and slot 623 limits the suture 710 from becoming entangled and/or pinched between the inner shaft 625 and outer shaft 620 when the anchor 700 and inner shaft 625 are rotated.

Referring to FIGS. 38A-39, the anchor 700 includes an anchor body 701 having exterior threads 740, such as helical screw threads, two suture holes 702, 703, and the suture 710 coupled to the anchor 700. In addition, the anchor body 701 defines a hollow cavity 704 and an exterior of the anchor body 701 which each taper distally to a smaller outer diameter at a distal portion 701b of the anchor body 701. Suture hole 703 is an opening formed in the proximal portion 700a of the anchor 700, e.g., the opening is the proximal portion of the cavity 704, which permits the assembler to tie the sliding knot 730 and place to position the sliding knot 130 within the cavity 704. Suture hole 702 extends at an angle with respect to suture hole 703 and the cavity 704, e.g., normal to the suture hole 703. The loop portion 720 of the suture 710 passes through the suture hole 702 and the tension portion 715 passes through the suture hole 703, e.g., and up through the inner shaft 625 when loaded in the delivery device 601. The loop portion 720 is wrapped around the inner shaft 625 (not shown in FIG. 39) tension portion 715 to form the wrapped portion 716.

The suture 710 includes a trailing end 708 that is secured to the anchor 700 at a distal portion 705 of the anchor 700, e.g., with a knot 708a. The leading end 718 of the suture 710 is passed through the cavity 704 within the anchor body and suture holes 702, 703 to form a sliding knot 730, the tension portion 710 and the loop portion 720. The suture 710 is movable through the anchor body 701 in a first direction, e.g., distally, while movement in a second, opposite direction, e.g., proximally, can be restricted by the sliding knot 730, such as a slip knot. Accordingly, tensioning of the leading end 718 of the tension portion 715 will result in tightening of the sliding knot 730 and reduction in the size of the loop portion 720. As with the anchor 100 described in connection with FIGS. 1-9, the sliding knot 730 remains suspended within the cavity 704 of the anchor 700 when the suture 710 has been tightened and secured, e.g., upon being secured within a repaired tissue. Since the anchor 700 is not provided with a plug, but instead includes a monolithic body, the suture sliding knot 730 is tied exterior to the anchor cavity 704 and drawn into the cavity when securing the knot 708a and/or by tensioning the leading end 718 of the suture 710.

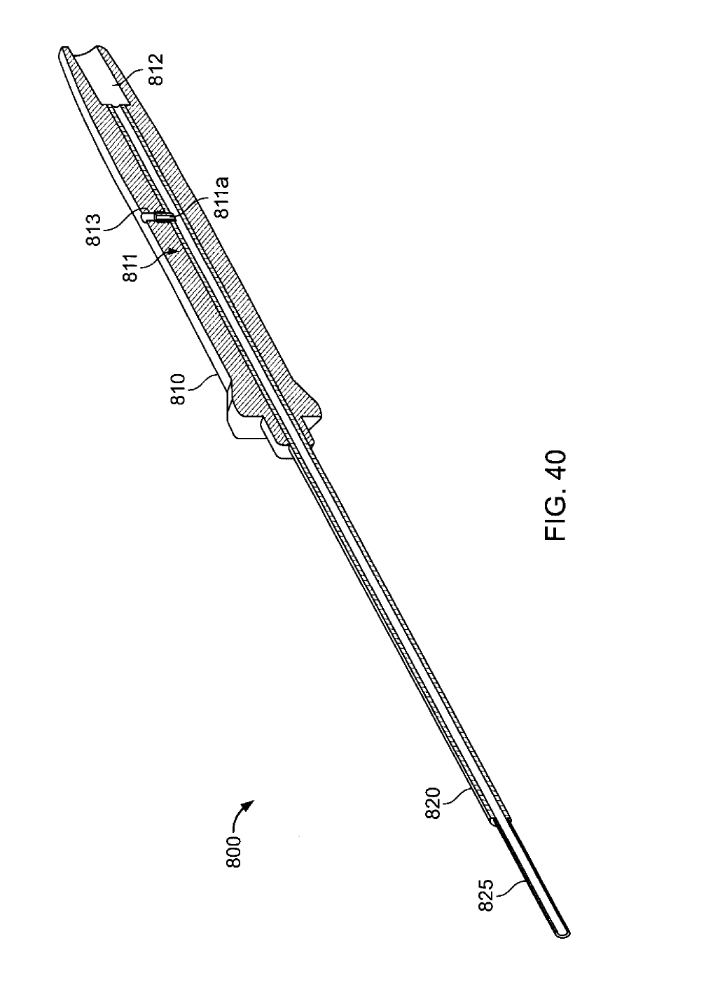

Referring to FIG. 40, an exemplary anchor delivery device 800 includes a handle 810, a stationary outer tube 820 and an axially movable inner tube 825, e.g., for axial advancement of an anchor (not shown). The handle 810 includes a longitudinally extending recess 812 for accommodating a suture puller (not shown) and a laterally extending bore 813 for accommodating a set screw 811. The outer shaft 820 is secured to the handle 810 by driving the set screw 811 into an opening in an outer surface of the outer shaft 820. The screw 811 includes a leading end 811a which protrudes through the opening in the outer surface of the outer shaft 820 and abuts against the inner shaft 825, e.g., the inner shaft 825 can include a longitudinally flat portion similar to the recessed, longitudinally flat portion 226 described in connection with FIG. 2. The leading end 811a of the screw serves as a key that permits the inner shaft 825 to move axially along the length of the longitudinally flat portion 226.

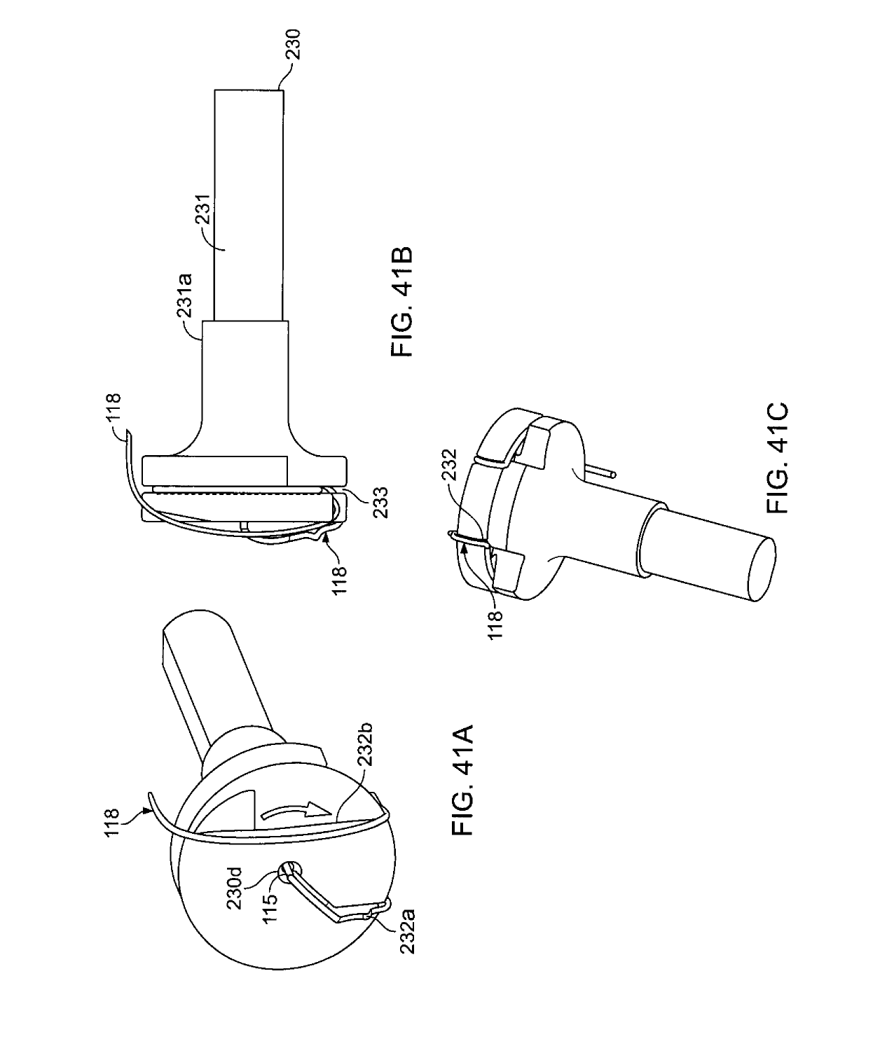

Referring to FIGS. 41A-41C, the suture puller 230 of FIG. 2 includes a circumferentially extending groove 233 and laterally extending grooves 232a, 232b formed in the proximal portion 230a of the suture puller 230. The depth stop 231a includes a raised edge larger in diameter than a shaft portion 231 of the suture puller 230 for limiting axial movement of the suture puller 230. A suture 115 which has been passed through a proximal opening 230d and distal opening 230c of a lumen of the suture puller 230 is secured to the circumferentially extending groove 233 and laterally extending grooves 232a, 232b. For example, the leading end 118 of the suture is passed out of the proximal end 230c of the lumen of the suture puller and through the first laterally extending groove 232a, is wrapped around the circumferentially extending groove 233, and is secured in the second laterally extending groove 232b. Accordingly, the leading end 118 of the suture 115 is stored in an accessible position for retrieval by a surgeon when the suture puller 230 is ready to be removed 230. The laterally extending grooves 232 are formed with a depth sufficient to protect the suture 115 from being struck when a surgeon strikes the proximal portion 230a of the suture puller 230 with a surgical mallet.

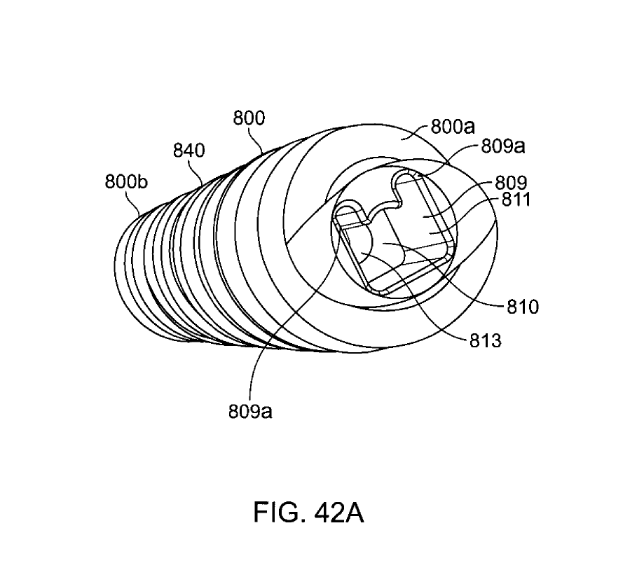

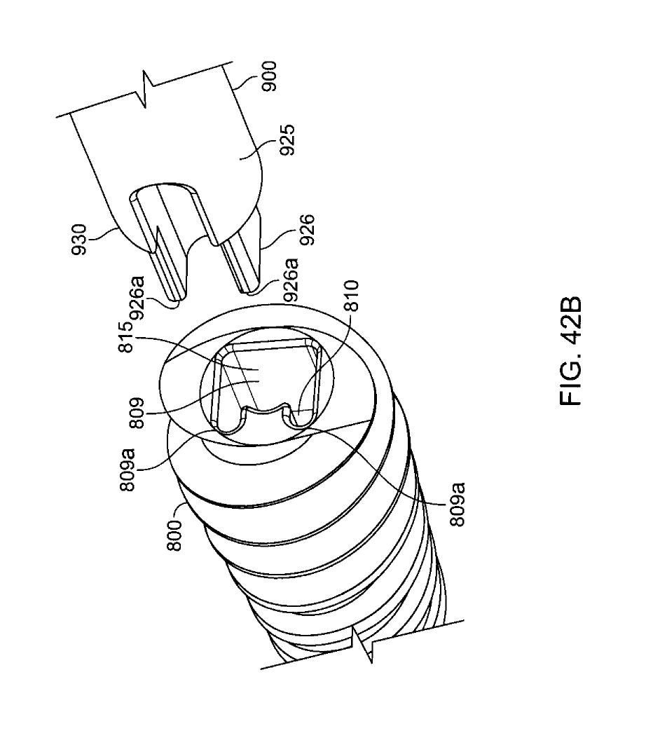

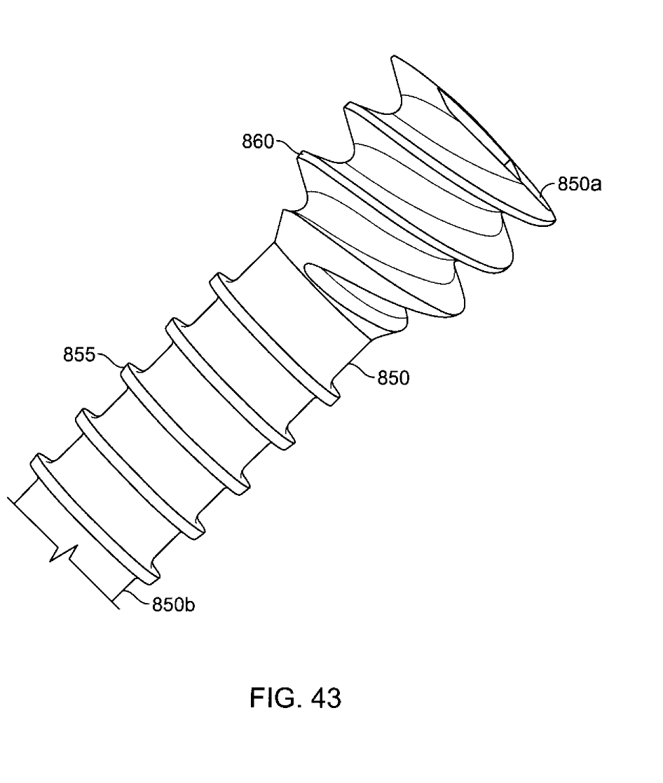

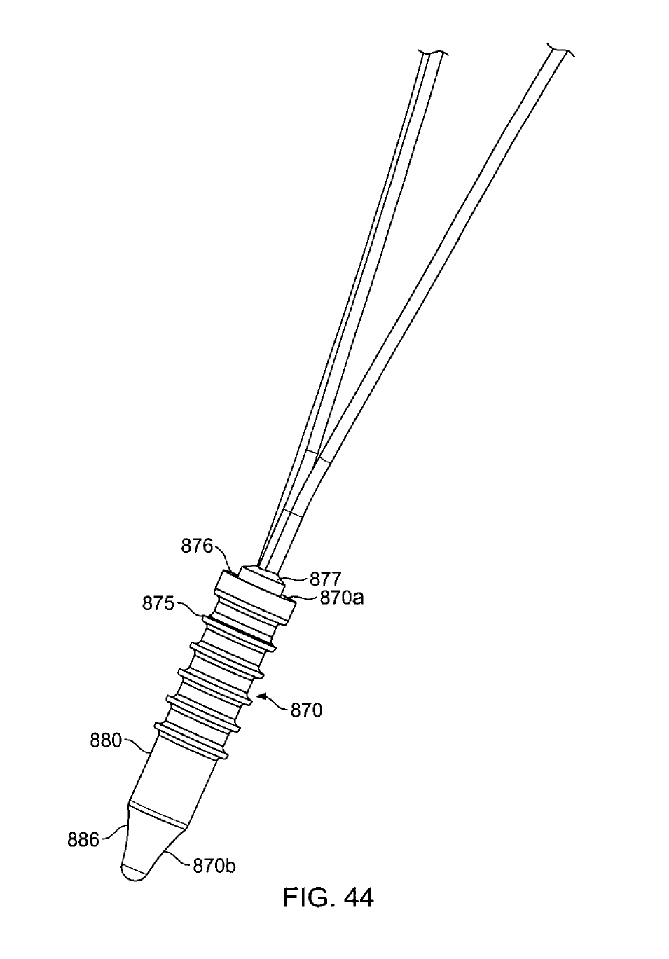

Referring to FIGS. 42A-B, an anchor 800 includes helical screw threads 840 between a proximal portion 800a and a distal portion 800b of the anchor 800. The proximal portion 800a of the anchor 800 defines a suture hole 809 that also serves as a drive recess for receiving an anchor deliver device 900. The anchor delivery device 900 includes a shaft 925, e.g., such as an axially or rotationally movable shaft, for driving the anchor 800 into a bone hole. The shaft 925 can be a movable inner or outer shaft similar to those described in the foregoing anchor delivery devices 200, 300, 400, 600 and 800. Alternatively, the shaft 925 can be affixed directly to a handle that can be rotated by a surgeon together with the shaft 925. The shaft 925 includes a U-shaped anchor drive portion 926 defining elongated arms 926a for engaging the proximal portion 800a of the anchor 800, and the suture hole 809 has a generally rectangular opening 811 and a pair of arm portions 809a that receive the elongated arms 926a, respectively. The shaft 925 includes an annular shoulder 930 which abuts against the proximal portion 800b of the anchor 800 when the drive portion 926 is inserted into the suture hole 809. Alternatively, or in addition, the suture hole 809 is provided with a bottom surface 810 extending partially across the interior of the suture hole 809 which acts as a land to prevent the insertion of the drive portion 926 beyond a desired depth into the anchor 800.

A suture (not shown), e.g., including a sliding knot, suture loop portion and tensioning portion, is positioned within the anchor 800 and is passed through an aperture 813 (FIG. 42A) within the bottom surface 810 and out of the suture hole 809. The U-shaped drive surface 926 permits greater surface to surface contact between shaft 925 and the anchor 800, and thereby permits greater loads to be applied to the anchor, e.g., such as a greater torsional or longitudinally oriented load to drive the anchor 800 into bone without stripping the suture hole 809. The anchor 800 is also held in a stable delivery position by the receipt of the drive portion 926 within the suture hole 809.

Referring to FIG. 43, an alternative anchor 850 for use with the aforementioned anchor delivery device 900 includes a suture hole like that of anchor 800. However, the anchor 850 includes a combination of circumferentially extending ribs 855 and helical screw threads 860 between a proximal portion 850a and distal portion 850b of the anchor 850. The anchor 850 can include various combinations of ribs and threads arranged to engage various tissue conditions typically encountered when repairing tissue. For example, the circumferentially extending ribs are provided at the distal portion 850b of the anchor 850 to engage relatively soft or degraded tissue found deeper within a bone hole. The helical screw threads 860 are provided at the proximal portion 850a of the anchor 850 to engage with relatively harder or stronger tissue within a bone hole. For example, an exemplary anchor has an overall length of approximately 0.625 inches, a ribbed section extending approximately 0.30 to 0.35 inches along the anchor length, and a threaded section extending approximately 0.18 to 0.20 inches along the anchor length. The anchor 850 can be provided with any number of ribs 855, e.g., five ribs, and/or threads, e.g., two or three screw turns.

Referring to FIG. 44, an alternative anchor 870 similar to the aforementioned anchor 500 includes circumferentially extending ribs 875 between a proximal portion 870a and a distal portion 870b thereof. The proximal portion 870a also includes an annular drive recess 876 and a single suture hole 877 for engaging with the drive portion of an anchor delivery device, such as anchor delivery device 400. However, the distal portion 870b includes a tapered tip 881, such as an anchor plug, and a relatively smooth surface 880 formed without ribs which eases the insertion and alignment of the anchor 870 into a bone hole that has been previously drilled in a tissue repair site. For example, the smooth surface 880 extends approximately 0.20 to 0.26 inches along the length of the anchor and away from the distal tip of the anchor. In addition, one or more of the aforementioned anchors can be provided with a smooth surface, e.g., without ribs and/or threads, at the distal portion of the anchor to assist the surgeon in the alignment of the anchor with a bone hole.

A number of embodiments have been described. Nevertheless, it will be understood that various modifications may be made. For example, one or more of the inner shaft and/or the outer shaft of an anchor delivery device can be retractable or rotatable with respect to the handle. The inner shaft can be movable with respect to a stationary outer shaft, or the outer shaft can be movable with respect to the inner shaft. The anchors may include one or more suture holes within various portions of the anchor body. The anchor bodies need hot include plugs.

The anchors used with any of the aforementioned anchor delivery devices can include any combination of one or more threads and/or one or more ribs. The threaded anchors can be inserted with either a rotary or an axially-oriented advancement force. The anchors can include various combinations of screw-in threads, circumferentially-extending ribs, and/or truncated and stacked conically shaped ribs. The anchors can be inserted with an axially oriented advancement force, such as by tapping with a surgical mallet and/or hand pressure, and/or a rotary advancement force, such as a rotary force imparted to an anchor including one or more screw threads.

The distal end of a suture puller can be press-fit or interference fit to the proximal end of the inner shaft of the anchor delivery device. The distal end of the suture puller can be removable with respect to the handle and the inner shaft. The suture puller can also be formed integrally as one piece or secured to each other, such as with an adhesive so that removal of the suture puller results in removal of the inner shaft. Alternatively, the distal end of the suture puller can abut against the proximal end of the inner shaft and not be secured to the inner shaft.

Although the foregoing embodiments have been described in connection with respect to a ball and socket joint, e.g., such as a glenoid-humeral joint or hip joint, the anchors and anchor delivery devices described above can be applied to other portions of the human anatomy, such as any area where relatively soft and/or fibrous tissue is to be repaired relative to an adjacent bony surface.

Although the suture has been described as being formed of a single piece of suture, the suture can include more than one piece of suture to form the combination of the tensioning portion, loop portion and sliding knot.

What is claimed is:

1. A surgical device comprising:
an anchor body defining a cavity and an opening to the cavity; and
a flexible member extending through the opening and having a sliding knot disposed within the cavity.

2. The surgical device of claim 1, wherein the anchor body includes a portion defining the opening and the cavity, and a plug received by the portion.

3. The surgical device of claim 1, wherein the sliding knot comprises a slip knot.

4. The surgical device of claim 1, wherein the sliding knot is configured such that the flexible member is movable through the anchor body in a first direction while movement of the flexible member in a second, opposite direction is restricted.

5. The surgical device of claim 1, wherein the opening comprises a through hole.

6. The surgical device of claim 5, wherein the anchor body defines at least two through holes.

7. The surgical device of claim 6, wherein the anchor body defines at least three through holes.

8. The surgical device of claim 1, wherein the anchor body is configured for rotary advancement into a target tissue.

9. The surgical device of claim 8, wherein the anchor body includes screw threads.

10. The surgical device of claim 1, wherein the anchor body is configured for axially-oriented advancement into a target tissue.

11. The surgical device of claim 10, wherein the anchor body includes a circumferential rib.

12. The surgical device of claim 1, wherein the flexible member comprises a single suture forming a loop portion, a tension portion and the sliding knot.

13. The surgical device of claim 1, further comprising:
an outer shaft, wherein the anchor body is received within the outer shaft; and
an inner shaft received within the outer shaft.

14. The surgical device of claim 13, further comprising a handle, wherein the outer shaft is attached to the handle.

15. The surgical device of claim 13, further comprising a handle, wherein the outer shaft is movable with respect to the handle.

16. The surgical device of claim 13, wherein the inner shaft is arranged for engagement with a proximal portion of the anchor body.

17. A surgical device comprising:
an outer shaft defining a longitudinally extending slot; and
an inner shaft received within the outer shaft, wherein the inner shaft defines a recess associated with the slot of the outer shaft such that when an anchor with a flexible member coupled thereto is received within the outer shaft, the flexible member passes through the recess and the slot.

18. The surgical device of claim 17, further comprising the anchor.

19. A method for repairing a soft tissue, comprising:
placing an anchor in bone, the anchor having a flexible member coupled thereto in the form of a loop;
passing the loop through an opening in the tissue;
positioning the loop around the anchor; and
tensioning the flexible member to decrease a size of the loop to secure the tissue to the bone.

20. The method of claim 19, wherein positioning the loop comprises passing the anchor through the loop and into the bone.

21. The method of claim 19, wherein positioning the loop occurs after placing the anchor.

22. A method of forming an anchor, comprising:
providing an anchor body defining a cavity and an opening to the cavity,
passing a flexible member through the opening to the cavity; and
forming a sliding knot in the flexible member, the knot being positioned within the cavity.

23. A tissue fixation member for use in a surgical procedure, comprising:
a body,
a flexible member, and
means for coupling the flexible member to the anchor,
the tissue fixation member configured such that without having to tie a knot in the flexible member during the surgical procedure, the flexible member is movable through the body in a first direction and movement of the flexible member in a second, opposite direction is restricted.
